(19) United States
(12) Patent Application Publication
(10) Pub. No.: US 2015/0285807 A1
(43) Pub. Date: Oct. 8, 2015

(54) SYSTEM AND METHOD FOR DETECTING CANCER

(71) Applicant: THE BRIGHAM AND WOMEN'S HOSPITAL, INC., Boston, MA (US)

(72) Inventors: Yujiang Geno Shi, Chesnut Hill, MA (US); Christine Guo Lian, Shrewsbury, MA (US)

(73) Assignee: THE BRIGHAM AND WOMEN'S HOSPITAL, INC., Boston, MA (US)

(21) Appl. No.: 14/405,673

(22) PCT Filed: Jun. 11, 2013
(86) PCT No.: PCT/US2013/045227

§ 371 Date: Dec. 4, 2014

Related U.S. Application Data
(60) Provisional application No. 61/689,739, filed on Jun. 11, 2012.

(51) Int. Cl. G01N 33/574; C12Q 1/68; A61K 38/44

(52) U.S. Cl. CPC G01N 33/57484; A61K 38/443; A61K 38/44; C12Q 1/6804; C12Q 1/6886; C12Q 2600/154; G01N 2400/00; C12Y 101/01042; C12Y 114/11; A61K 48/00

(57) ABSTRACT

Disclosed herein are methods of detecting malignancy in a tumor of a subject comprising measuring the amount of 5-hmC in a tumor sample and comparing to a control to thereby detect a level of reduction of 5-hmC in the tumor, wherein the tumor is characterized as malignant if a threshold level of reduction is detected. The tumor may be a solid tumor such as a melanocytic lesion. Also disclosed are methods of determining the prognosis of a subject with a tumor by determination of the amount of 5-hmC in the tumor. Methods for treatment of a tumor with reduced 5-hmC are also disclosed.

### RELATED APPLICATIONS

This application claims the benefit under 35 U.S.C. §119(e) of U.S. Provisional Application Ser. No. 61/689,739, filed Jun. 11, 2012, the contents of which are incorporated herein by reference in their entirety.

### GOVERNMENTAL SUPPORT

This invention was made with Government support under GM078458 and 5P50CA093683-08 awarded by the National Institutes of Health. The Government has certain rights in the invention.

### FIELD OF THE INVENTION

The present invention relates to the field of cancer diagnosis and therapeutics.

### BACKGROUND OF THE INVENTION

Melanoma is a unique, highly aggressive type of cancer, which occurs more frequently with increasing age and often with a significant contribution of environmental factors to its etiology (Jemal et al., 2001; Jemal et al., 2006; Marks, 2000). As one of the most virulent human cancers, melanoma is capable of distant and lethal metastases when the primary tumor volume is as little as 1 mm³. Studies of biomarkers predictive of clinical outcome are impeded by latent periods for detection of metastases that may range from several years to more than a decade, and thus clinically-annotated bio-specimen archives serve as valuable surrogates for the otherwise impractical prospective approaches. Such studies are further compounded by the difficulties inherent in the diagnosis of melanoma, since certain benign nevi and melanomas show significant histologic overlap. Presently, there is a dearth of molecular markers that facilitate detecting the differences between benign and malignant melanocytic lesions and assist in predicting their biological behaviors. Thus, there is a pressing need for novel biomarkers that define the malignant potential of primary lesions, predict clinical outcome, and forecast therapeutic responses.

Abnormal DNA methylation at the 5-position of cytosine (5-mC) is a well-known epigenetic feature of cancer. Melanoma exhibits global hypomethylation within the bulk genome and local hypermethylation at specific tumor suppressor genes (Hoon et al., 2004; Liu et al., 2008; Shen et al., 2007). Nonetheless, the degree of global hypomethylation in melanoma is not sufficient to distinguish benign nevus from melanoma (Paz et al., 2003). Gene-specific hypermethylation may be a better discriminator as recent studies indicate that multi-locus DNA-methylation signature genes may differentiate melanomas from nevi (Conway et al., 2011; Tellez et al., 2009). However, this requires sophisticated molecular biological tools that are not easily applicable in routine clinical practice, and the small biopsy size of melanocytic lesions presents another technical limitation. Thus, despite the increasing recognition that abnormal DNA methylation (and/or histone modification) is a crucial participant in melanoma progression; no characteristic epigenetic modifications have been discovered that can be readily used as molecular markers for diagnosis and evaluation of melanoma virulence.

The recent discovery of the Ten-Eleven Translocation (TET) family of 5-mC hydroxylases, including TET1, 2 and 3, which convert 5-mC to 5-hydroxymethylcytosine (5-hmC), also known as the “sixth base”, has added an additional layer of complexity to the epigenetic regulation of DNA methylation (Ito et al., 2010; Tahiliani et al., 2009; Zhang et al., 2010). 5-hmC exists at a high level in self-renewing and pluripotent stem cells (Szwagierczak et al., 2010; Tahiliani et al., 2009). However, 5-hmC levels are greatly reduced in most cultured, immortalized tumor cells (Haffner et al., 2011; Song et al., 2011; Yang et al., 2012). Frequent TET2 mutational inactivation has been reported to associate with decreased 5-hmC levels in various myeloid leukemias (Delhommeau et al., 2009; Langemeijer et al., 2009). In addition, the co-factor α-ketoglutarate (α-KG) is absolutely required and plays a positive and critical role in the conversion of 5-mC to 5-hmC (Xu et al., 2011a). Isocitrate dehydrogenases (IDHs) catalyze oxidative decarboxylation of isocitrate, producing α-KG and CO₂ (Reitman et al., 2011; Xu et al., 2011a). There are two major IDH enzymes in mammalian cells, IDH1 in cytoplasm and its homologue, IDH2, in mitochondria, which catalyze the same reaction. It has been reported that gain-of-function mutations in IDH1 and IDH2 in cancer cells produce the oncometabolite 2-hydroxyglutarate (2-HG), an antagonist of α-KG (Chowdhury et al., 2011; Xu et al., 2011a), which inhibits the TET-mediated conversion of 5-mC to 5-hmC. Moreover, similar to the frequent mutation rate of IDH1 or IDH2 in glioma and myeloid leukemia (Dang et al., 2010; Krell et al., 2011), 10% of melanomas harbor a neomorphic mutation in IDH1 or IDH2 (Shibata et al., 2011). These studies suggest a role of 5-hmC, TET and IDH in malignancy. However, it remains elusive as to how 5-hmC is lost and what roles TET and IDH proteins play during tumor progression. In particular, it remains unknown as to how this epigenetic mark and these related enzymes partake in melanoma progression.

### SUMMARY OF THE INVENTION

### Definitions

The term “treating” or “successfully treating” when used in the context of treating melanoma, including metastatic melanoma, shall include shrinking a tumor, curing melanoma, including melanoma which has metastazied (by causing a remission of the cancer in the patient) or reducing the likelihood or preventing the spread of the melanoma into other organs. Melanoma, including metastatic melanoma, may be treated using compounds alone, or in combination with other methods and/or compounds including surgery, chemotherapy (especially the use of the chemotherapeutic agent dacarbazine or DTIC), radiation therapy and immunotherapy (IL-2 and/or alpha-interferon).

Treatment refers to a therapeutic intervention that ameliorates a sign or symptom of a disease or pathological condition related to a disease (such as skin cancer). Treatment can also induce remission or cure of such condition. In particular examples, treatment includes inhibiting a tumor, for example by inhibiting the full development of a tumor, such as preventing development of a metastasis or the development of a primary tumor, reducing tumor volume, or reducing the total number of tumors. Inhibition may not require a total absence of a tumor. In other examples, treatment includes inhibiting, reducing the risk of, or delaying development of, skin cancer. Reducing or suppressing a sign or symptom associated with a disease (such as a tumor, for example, skin cancer) can be evidenced, for example, by a delayed onset of clinical symptoms of the disease in a susceptible subject (such as a subject having a tumor which has not yet metastasized), a reduction in severity of some or all clinical symptoms of the disease, a slower progression of the disease (for example by prolonging the life of a subject having the disease), a reduction in the number of relapses of the disease, an improvement in the overall health or well-being of the subject, or by other parameters well known in the art that are specific to the particular disease.

The term “tumor”, as used herein refers to an abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The term “patient” or “subject” or “individual” is used throughout the specification to describe an animal, preferably a human, to whom treatment, including prophylactic treatment, with the compounds according to the present invention is provided. For treatment of those infections, conditions or disease states which are specific for a specific animal such as a human patient, the term patient refers to that specific animal.

Aspects of the invention relate to a method of detecting malignancy in a tumor of a subject comprising, processing a sample of the tumor to thereby label the 5-hmC present in the sample, measuring the amount of 5-hmC in the sample by detection of the labeled 5-hmC in the sample, quantitating the amount of 5-hmC in the sample as compared to a healthy control to thereby detect a threshold level of reduction of 5-hmC in the sample, and characterizing the tumor as malignant when a threshold level of reduction of 5-hmC is detected. In one embodiment of the herein described methods, the tumor is selected from the group consisting of a breast tumor, a colon tumor, skin tumor, ovarian tumor, lung tumor, liver tumor, prostate tumor, brain tumor, and kidney tumor. In one embodiment of the herein described methods the processing step is by immunohistochemical staining or by immunofluorescence.

Another aspect of the invention relates to a method of diagnosing a subject with a melanocytic lesion comprising, processing a tissue sample of the melanocytic lesion of the subject to thereby label the 5-hmC present in the tissue sample, measuring the amount of 5-hmC in the sample by detection of the labeled 5-hmC in the tissue sample, quantitating the amount of 5-hmC in the tissue sample as compared to a healthy control to thereby detect a threshold level of reduction of 5-hmC in the sample, and characterizing the melanocytic lesion by the detected level of reduction of 5-hmC to thereby diagnose the subject. In one embodiment, the processing step is by immunohistochemical staining or by immunofluorescence. In one embodiment, quantitating is by assignment of a 5-hmC staining score. In one embodiment of the herein described methods, the threshold level of reduction is equivalent to a 5-hmC staining score of ≦2 which indicates the melanocytic lesion is a stage 2-3 melanoma, with a Breslow of >1 mm, and >1 mitosis, a 5-hmC staining score of ≧3 which indicates the melanocytic lesion is a stage 1 melanoma, with a Breslow of ≦1 mm, and ≦1 mitosis, a 5-hmC staining score of ≧1.5 which indicates the melanocytic lesion is a melanoma without ulceration, or a 5-hmC staining score of <1.5 which indicates the melanocytic lesion is a melanoma with ulceration. In one embodiment of the herein described methods quantitating step is by assignment of a 5-hmC cell count score. In one embodiment of the herein described methods, the threshold level of reduction is equivalent to a 5-hmC cell count score of ≧2 which indicates the melanocytic lesion is a benign melanocytic nevus, a 5-hmC cell count score of ≧3 which indicates the melanocytic lesion is a benign melanocytic nevus, a 5-hmC cell count score of <1 which indicates the melanocytic lesion is a primary cutaneous melanoma or a visceral metastatsic melanoma, a 5-hmC cell count score of <0.5 which indicates the melanocytic lesion is a primary cutaneous melanoma or a visceral metastatsic melanoma, or a 5-hmC cell count score of <0.25 which indicates the melanocytic lesion is a lymphnode metastatic melanoma. In one embodiment of the herein described methods processing is by purifying genomic DNA from a tissue sample of the melanocytic lesion and measuring is by sequencing genomic DNA identified as containing 5-hmC in the purified genomic DNA. In one embodiment of the herein described methods processing is by purifying genomic DNA from a tissue sample of the melanocytic lesion and measuring is by detection of the 5-hmC in specific genes of the genomic DNA. In one embodiment, the threshold level of reduction is a ≧5 fold reduction in 5-hmC of the specific genes of the genomic DNA which indicates the melanocytic lesion is a melanoma. In one embodiment of the herein described methods processing step is by purifying genomic DNA from a tissue sample of the melanocytic lesion and measuring is by an anti-5-hmC antibody-based detection system. In one embodiment, the anti-5-hmC antibody-based detection system is a dot blot assay. In one embodiment the 5-hmC levels are detected by a 5-hmC glucosylation assay. In one embodiment, the 5-hmC glucosylation assay is a T4 phage β-glucosyltransferase-mediated 5-hmC glucosylation assay.

Another aspect of the invention relates to a method of diagnosing a subject having a melanocytic lesion comprising, processing a tissue sample of the melanocytic lesion of the subject to thereby label the expression product of one or more of the genes IDH2, TET1, TET2, TET3, measuring the amount of the expression product of the one or more genes in the sample by detection of the labeled expression product in the tissue sample, quantitating the amount of labeled expression product(s) in the sample as compared to a healthy control to thereby detect a threshold level of reduction of the expression product(s) in the sample, and diagnosing the subject as having a malignancy if a level of reduction of TET3 and/or IDH2 of >50% is detected, and/or if a level of reduction of TET1 and/or TET2 of >75% is detected.

Another aspect of the invention relates to a method of determining prognosis of a subject with a melanocytic lesion comprising, processing a tissue sample of the melanocytic lesion of the subject to thereby label the 5-hmC present in the tissue sample, measuring the amount of 5-hmC in the sample by detection of the labeled 5-hmC in the tissue sample, quantitating the amount of 5-hmC in the tissue sample as compared to a healthy control to thereby detect a threshold level of reduction of 5-hmC in the sample, correlating the detected threshold level of reduction with one or more melanoma staging parameters, and determining the prognosis of the subject based on that of the staging parameter to which the amount of 5-hmC correlates. In one embodiment, the staging parameter is selected from the group consisting of Breslow depth, mitosis rate, presence or absence of ulceration, overall stage of melanoma, melanocytic lesion type, and combinations thereof. In one embodiment, processing is by immunohistochemical staining or by immunofluorescence. In one embodiment, quantitating is by assignment of a 5-hmC staining score. In one embodiment of the various inventions disclosed herein, the threshold level of reduction is equivalent to a 5-hmC staining score of ≦2 which indicates the melanocytic lesion is a stage 2-3 melanoma, with a Breslow of >1 mm, and >1 mitosis, a 5-hmC staining score of ≧3 which indicates the melanocytic lesion is a stage 1 melanoma, with a Breslow of ≦1 mm, and ≦1 mitosis, a 5-hmC staining score of ≧1.5 which indicates the melanocytic lesion is a melanoma without ulceration, or a 5-hmC staining score of <1.5 which indicates the melanocytic lesion is a melanoma with ulceration. In one embodiment of the invention quantitating step is by assignment of a 5-hmC cell count score. In one embodiment of the various inventions disclosed herein the threshold level of reduction is equivalent to a 5-hmC cell count score of ≧2 which indicates the melanocytic lesion is a benign melanocytic nevus, a 5-hmC cell count score of ≧3 which indicates the melanocytic lesion is a benign melanocytic nevus, a 5-hmC cell count score of <1 which indicates the melanocytic lesion is a primary cutaneous melanoma or a visceral metastatsic melanoma, a 5-hmC cell count score of <0.5 which indicates the melanocytic lesion is a primary cutaneous melanoma or a visceral metastatsic melanoma, or a 5-hmC cell count score of <0.25 which indicates the melanocytic lesion is a lymphnode metastatic melanoma.

Another aspect of the invention relates to a method for treating a subject with a melanoma comprising contacting melanoma cells of the subject with an effective amount of an agent that increases expression of IDH2 and/or TET2 sufficient to increase 5-hmC in the genome of the cell. In one embodiment the agent is selected from an expression vector encoding IDH2 and/or TET2, a regulatory molecule which increases transcription or translation of the IDH2 and/or TET2 gene, and combinations thereof. In one embodiment contacting is by administering to the subject a therapeutic amount of a pharmaceutical composition comprising the agent. In one embodiment, administering is intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, or intratumor.

### BRIEF DESCRIPTION OF THE DRAWINGS

FIG. 1A-FIG. 1J show experimental results that indicate 5-hmC level is high in mature melanocytes and lost in melanomas. (A-B) IF co-staining of 5-hmC and MART1 in normal human skin without HCl treatment. 5-hmC was visualized in green, MART1 was visualized in red; DAPI counterstain of DNA was visualized in blue. Among basal layer cells (thin dotted line), 5-hmC-positive cells exclusively proved to be MART-1-positive melanocytes (dotted circles). (C-D) Detecting 5-hmC in normal human skin by IF (C) and IHC staining (D) with HCl treatment. Both methods showed strong nuclear staining in isolated, solitary cells within the basal layer (dotted circles), in nuclei within the uppermost epidermal layers and occasional dermal cells. (E-H) Representative histology of 5-hmC IHC staining in the individual cases of benign and malignant melanocytic lesions. Low power images (100×) on the left column with the dotted area magnified at high power (400×) on the right column. All slides were counterstained with hematoxylin to visualize in light blue. (I) Immunoblotting assay shows significantly higher 5-hmC levels in benign nevi than in melanomas. Three representative immunoblot images are shown here from the 10 cases of each group. (J) 5-hmC glucosylation assay confirms that the 5-hmC level in the genomic DNA of nevi is significantly higher than that in melanomas. **P<0.01 by Student's t-test. Data are shown as mean±SD (n=3). See also FIG. 7 and Table 1.

FIG. 2A-FIG. 2F show experimental results that indicate loss of 5-hmC correlated with melanoma progression. (A) Analysis of 5-hmC levels in the SPORE TMA represented by positive cell count score. Each column represents a category of melanocytic lesion (n=number of cases, each case has duplicated tissue cores). Data are shown as mean±SEM. ***P<0.001 compared to benign thin nevi; #P<0.001 compared to benign thick nevi. (B) Combined cell count scores of 5-hmC staining of three tissue microarrays. Each column represents a category of melanocytic lesion (n=number of cases). Data are shown as mean±SEM. ***P<0.001 compared to benign nevus, #P<0.05 compared to visceral metastases. (C-D) The Spearman correlation between Breslow depth and 5-hmC staining product score (C) or between mitosis and 5-hmC staining product score (D). (E) 5-hmC staining product scores are correlated with critical melanoma staging parameters. Data are shown as mean±SEM. *P<0.05, **P<0.01 by Student's t-test. (F) Kaplan-Meier survival curves of melanoma patients with positive 5-hmC staining (solid line) and negative 5-hmC staining (dashed line). P<0.05 by Gehan-Breslow-Wilcoxon Test. See also FIG. 8 and Tables 2-6.

FIG. 3A-FIG. 3G show experimental results that indicate genome-wide mapping of 5-mC and 5-hmC in benign nevi and melanomas. (A) The distribution of 5-mC (visualized in green) and 5-hmC (visualized in blue) densities in the region of chr16:46,651,039-89,749,255 by MeDIP-seq and hMeDIP-seq. Refseq genes are shown at the bottom. (B) 5-mC and 5-hmC peak numbers of nevus (red) and melanoma (blue) hMeDIP samples in different genomic regions. Promoters were defined as −2k to +2k relative to TSS. (C-D) Normalized 5-hmC (C) and 5-mC (D) tag density distribution across the gene body. Each gene body was normalized to 0-100%. Normalized Tag density is plotted from 20% of upstream of TSSs to 20% downstream of TTSs. Note, the top line of the graph indicates the results for the nevus. (E) Peaks at which 5-hmC is significantly reduced (>5-fold) and 5-mC is significantly increased (>2-fold) in gene bodies in melanomas (Mel) compared to nevi (left panel), and the KEGG pathway analysis results for the associated genes (right panel). (F-G) MeDIP-seq and hMeDIP-seq results of RAC3, IGF1R and TIMP2 genes (F) and hMeDIP-qPCR verifications (G). The primer targeted regions in panel G are noted by red lines in panel F. Data are shown as mean±SD (n=3) in panel G. See also FIG. 9 and Table 7.

FIG. 4A-FIG. 4F show experimental results that indicate increased 5-hmC level by IDH2 over-expression in a zebrafish melanoma model prolongs tumor free survival. (A) Schematic diagram of 5-hmC generation by the TET family of 5-mC DNA hydroxylases with cofactors α-ketoglutarate and Fe²⁺. (B) Relative expression of genes in nevus and melanoma by RT-qPCR. Each gene expression level was normalized to HPRT house-keeping gene. Data are shown as the mean of three individual patients±SEM. *P<0.05, **P<0.01, ***P<0.001 by Student's t-test comparing nevus to melanoma. (C) Relative TET2 expression in human melanoma cDNA arrays including normal skin (n=3), stage III (n=21) and stage IV melanomas (n=19) by RT-qPCR. Data are shown as mean±SEM. ***P<0.001 compared the normal skin by Student's t-test. (D) Representative IDH2 IHC staining images in nevi (n=4) and melanomas (n=8) at high power (400×). (E) IF staining of 5-hmC and mitfa in normal zebrafish melanocytes. mitfa was visualized in green; 5-hmC was visualized in red; DAPI counterstain of DNA was visualized in blue. (F) Tumors were smaller and less invasive and had higher 5-hmC levels in miniCoopR IDH2 zebrafish than mini-CoopR EGFP control zebrafish. Histology of the melanomas from miniCoopR-EGFP control zebrafish and miniCoopR-IDH2 zebrafish are shown in the left panels. The H&E staining of tumor sections shows an infiltrative pattern of tumor in control miniCoopR-EGFP zebrafish at the body and tail junction, while the tumor shows much less infiltrative borders in IDH2 over-expressing zebrafish. (G) Significant prolongation of tumor-free survival in miniCoopR-IDH2 zebrafish (n=77) compared with miniCoopR-EGFP control zebrafish (n=125). See also FIG. 10 and Table 8.

FIG. 5A-FIG. 5H show experimental results that indicate TET2 over-expression re-establishes the 5-hmC landscape in the epigenome of human melanoma cells. (A) Schematic diagram of TET2 wt and TET2 catalytically inactive mutant (TET2 M) proteins. (B) The expression of Flag-tagged TET2 and Flag-tagged TET2 M proteins by Western blot. Red arrow denotes the full length TET2 and TET2 M bands, and red star denotes non-specific bands. ACTB was used as a loading control. (C) Global 5-hmC levels in MOCK, A2058 TET2 and A2058 TET2 M stable cell lines by dot-blot assay. The Methylene blue staining was used as total genomic DNA loading control. (D) IF analysis of A2058 TET2 and A2058 TET2 M stable cell lines. The Flag antibody was used to detect Flag-tagged TET2 and Flag-tagged TET2 M. DAPI counterstain of DNA visualized in blue; Flag visualized in green; 5-hmC visualized in red. (E) Normalized 5-hmC tag density distribution across the gene body. Each gene body was normalized to 0-100%. Normalized tag density is plotted from 20% of upstream of TSSs to 20% downstream of TTSs. Note that the Tet2 data is the top line, the mock data is the middle line, and the Tet2M is the bottom line. (F-G) MeDIP-seq and hMeDIP-seq results of CCND1 and MC1R genes (F) and hMeDIP-qPCR verifications (G). The primer targeted regions in panel G are noted by lines beneath the graphs in panel F. Data are shown as mean±SD (n=3) in panel G. (H) Venn diagrams showing the overlap between 5-hmC peaks which are dramatically higher (>5-fold) in nevi than melanomas (pink) and 5-hmC peaks which are dramatically higher (>5-fold) in TET2 over-expression cells compared to TET2 M over-expression cells (blue) (left panel), and the associated genes according the peak location either at gene promoter (middle upper panel) or in gene body (middle lower panel). The GO term and KEGG pathway analyses results are shown in the right panels. See also FIGS. 9 and 11 and Table 7.

FIG. 6A-FIG. 6E show experimental results that indicate over-expression of TET2 in human melanoma cells suppresses tumor growth in NSG xenograft mice. (A) The proliferation curves of A2058 TET2 and A2058 TET2 M stable cell lines. (B) A2058 TET2 melanoma cells show less in vitro invasion than A2058 TET2 M melanoma cells by Matrigel tumor invasion assay. Data are shown as mean±SD (n=3). **P<0.01 by Student's t-test.

(C) Tumor growth curves of A2058 TET2 and A2058 TET2 M cells xenografted to NSG mice. Data are shown as mean±SEM (n=10). *P<0.05, **P<0.01 by Student's t-test. (D) Representative images of tumor-bearing NSG mice xenografted with A2058 TET2 M (left) or A2058 TET2 cells (right) at 4 weeks post inoculation. (E) H&E and 5-hmC IHC staining of TET2 M (upper) and TET2 (lower) xenografts. The regions shown in left panels are noted by dash circles in panel D.

FIG. 7A-FIG. 7B show experimental results that indicate IHC staining of epigenetic marks in normal and cancer tissues, Related to FIG. 1 (A) 5-hmC IHC staining in normal human tissues. (B) IHC staining patterns of two representative epigenetic marks, H3K4 me2 and H3K36 me3 in benign nevus and melanoma. Nuclei of melanoma cells (lower panels) are typically 2-3 times bigger than those of benign nevus (upper panels).

FIG. 8 shows photographs of experimental results that indicate the scoring system of 5-hmC levels by IHC staining, Related to FIG. 2. Representative histology of the scoring system for cell counts of 5-hmC positive immunoreactivity. 0=Negative (<1% tumor cells immunoreactive); 1+=Low positive (<10% tumor cells immunoreactive); 2+=Positive (10-24% tumor cells immunoreactive); 3+=Positive (25-74% tumor cells immunoreactive) and 4+=Positive (>74% tumor cells immunoreactive).

FIG. 9A-FIG. 9D show experimental results that indicate genome-wide analyses of 5-mC and 5-hmC levels in nevus and melanoma, Related to FIGS. 3 and 5. (A) Summary of mapped reads numbers of each sequencing sample. (B) 5-mC and 5-hmC distributions in different genomic regions in nevus and melanoma. Note the promoters are represented by the bottom fragment of the bar, exons are represented by the fragment of the bar just above the promoter fragment, introns are represented by the fragment of the bar just above the intron fragment, and intergenic regions are represented by the top fragment of the bar. (C) Peaks at which 5-hmC is significantly reduced in melanomas (Mel) compare to nevi (>5-fold) and 5-mC is significantly increased (>2-fold) at gene promoters (left panel), and the KEGG pathway analysis result for the associated genes (right panel). (D) Representative gene promoters showing significantly reduced 5-hmC and increased 5-mC levels in melanoma compare to nevus.

FIG. 10 shows experimental results that indicate over-expression of IDH2, but not IDH2 R172K mutant, increases 5-hmC level and prolongs tumor free survival in a zebrafish melanoma model, Related to FIG. 4.

FIG. 11A-FIG. 11B show experimental results that indicate over-expression of TET2, but not TET2 M, re-establishes the 5-hmC landscape in human melanoma cells, Related to FIG. 5. (A) Over-expression of TET2 and TET2 M mRNA by RT-qPCR. Gene expression is normalized to HPRT. *: The over-expressed TET2 and TET2 M levels are normalized to the endogenous TET2 level in MOCK A2058 cells. Data shown as mean±SD (n=3). (B) Normalized 5-mC tag density distribution across the gene body. Each gene body was normalized to 0-100%. Normalized tag density is plotted from 20% of upstream of TSSs to 20% downstream of TTSs. Note that the Tet2 data is the top line, the mock data is the middle line, and the Tet2M is the bottom line.

### DETAILED DESCRIPTION OF THE INVENTION

DNA methylation at the 5-position of cytosine (5-mC) is a key epigenetic mark critical for various biological and pathological processes. 5-mC can be converted to 5-hydroxymethylcytosine (5-hmC) by the Ten-Eleven Translocation (TET) family of DNA hydroxylases. The experimental results reported herein indicate that the “loss of 5-hmC” is an epigenetic hallmark of melanoma with diagnostic and prognostic implications. Genome-wide mapping of 5-hmC revealed loss of the 5-hmC landscape in the melanoma epigenome. Down-regulation of Isocitrate Dehydrogenase 2 (IDH2) and TET family enzymes was shown as one of the mechanisms underlying 5-hmC loss in melanoma. Rebuilding the 5-hmC landscape in melanoma cells by reintroducing active TET2 or IDH2 is shown to suppress melanoma growth and increase tumor-free survival. These results indicate a critical function of 5-hmC in melanoma development and directly link the IDH and TET activity-dependent epigenetic pathway to 5-hmC-mediated suppression of melanoma progression, indicating a new strategy for epigenetic cancer therapy.

Aspects of the invention are based on the discovery that the levels of 5-hydroxymethyl cytosine (5-hmC) in a melanocytic lesion decreases as tumorigenicity and malignancy increases. Importantly, the findings of studies detailed herein indicate a correlation of quantitative levels of 5-hmC in a tissue sample with tumor type, tumor grade and tumor staging parameters. This indicates that the level of 5-hmC can be used as an epigenetic marker for diagnosis and evaluation of melanoma virulence. These findings can also be applied to other types of solid tumors.

### Detection of Malignancy and Diagnosis

One aspect of the invention relates to a method of detecting a malignancy in a tumor of a subject. The method comprises measuring the amount of the 5-hmC in the tumor and quantitating the amount of the 5-hmC in the tumor compared to a normal/healthy control. Detection of a pre-established threshold level of reduction of 5-hmC in the tumor indicates a specific level of malignancy of the tumor.

The amount of 5-hmC in the tumor is typically determined by obtaining a sample of the tumor and labeling the 5-hmC in the sample with a detectable label. The amount of the 5-hmC is then determined quantitatively by detection of the label. The amount of 5-hmC detected is compared to that of an appropriate control sample, which may be obtained by identical processing of the control or by comparing the level to a pre-established control amount. Appropriate controls can be obtained from the same individual (e.g., the same tissue type from a healthy source in the individual) or from a different individual. In one embodiment, the tumor is a melanocytic lesion and the control is a normal keratinocyte or melanocyte from the same subject.

A threshold level of reduction of 5-hmC for a specific lesion or tumor type is established by the guidance provided herein as exemplified for the melanocytic lesions and melanoma. Examples of specific thresholds for detection of different melanocytic lesion types are provided herein.

One aspect of the invention relates to a method of diagnosing a subject with a melanocytic lesion. The method comprises measuring the level of 5-hydroxymethyl cytosine (5-hmC) in a melanocytic lesion of the subject as compared to that of a normal control (e.g., a normal keratinocyte). Such measuring is performed to thereby quantitatively detect any decreased amount of 5-hmC in the melanocytic lesion as compared to that of the control. The decreased amount detected is then correlated to the melanocytic lesion type or tumor characteristic established for that decrease. The decreased amount can also or alternatively be correlated with staging parameters of malignancy of the lesion. Specific examples of correlation with melanocytic lesion types and staging parameters are provided herein. In one embodiment, the amount of 5-hmC is measured in a sample of the lesion, and the amount of reduction of 5-hmC in the sample is quantitated by comparison to a healthy control. The specific level of 5-hmC reduction compared to the control will indicate the characteristics of the lesion (e.g., stage, Breslow depth, mitotic rate, ulceration). Detection of a threshold level of reduction in 5-hmC in the sample as compared to the control indicates detection of the corresponding lesion characteristics. Specific threshold amounts are provided herein.

Measurement and quantitation of 5-hmC in a sample can be achieved by performing immunohistochemical staining of a melanocyte tissue sample (of formalin-fixed, paraffin embedded tissue sections) to detect 5-hmC. In one embodiment, the threshold level of reduction in 5-hmC in the sample corresponds to or is otherwise equivalent to a 5-hmC staining score obtained by the methods described herein (Table 2). Quantitating may be by assignment of a 5-hmC staining score or the equivalent value to the sample. Examples of threshold levels of 5-hmC reduction in a lesion correlates to a staining score are provided herein. In one embodiment, a 5-hmC staining score of ≦2 is used to indicate the melanocytic lesion is a stage 2-3 melanoma, with a Breslow of >1 mm, and >1 mitosis. In one embodiment, a 5-hmC staining score of ≧3 is used to indicate the melanocytic lesion is a stage 1 melanoma, with a Breslow of ≦1 mm, and ≦1 mitosis. In one embodiment, a5-hmC staining score of ≧1.5 is used to indicate the melanocytic lesion is a melanoma without ulceration. In one embodiment, a 5-hmC staining score of <1.5 is used to indicate the melanocytic lesion is a melanoma with ulceration. The skilled artisan will appreciate that different methods of detection and quantitation can be adapted from the staining score based methods disclosed herein.

Measurement and quantitation of 5-hmC in a sample can also be achieved by immunofluorescent staining in the nucleic of a melanocyte tissue sample (e.g., that coexpress MART-1 a melanocyte-specific marker) to detect 5-hmC. In one embodiment, the threshold level of reduction in 5-hmC in the sample corresponds to or is otherwise equivalent to a cell count score). Examples of threshold levels of 5-hmC reduction in a lesion correlated to a cell count score are provided herein (Table 2). Quantitating may be by assignment of a 5-hmC cell count score or the equivalent value to the sample. In one embodiment, a cell count score of ≧2 is used to indicate the melanocytic lesion is a benign melanocytic nevus. In one embodiment, a cell count score of ≧3 is used to indicate the melanocytic lesion is a benign melanocytic nevus. In one embodiment, a cell count score of <1 is used to indicate the melanocytic lesion is a primary cutaneous melanoma or a visceral metastatsic melanoma. In one embodiment, a cell count score of <0.5 is used to indicate the melanocytic lesion is a primary cutaneous melanoma or a visceral metastatsic melanoma. In one embodiment, al count score of <0.25 is used to indicate the melanocytic lesion is a lymphnode metastatic melanoma. The skilled artisan will appreciate that different methods of detection and quantitation can be adapted from the cell count score based methods disclosed herein.

Measurement and quantitation of 5-hmC in a sample can also be achieved by identifying and/or sequencing genomic DNA. In one embodiment, that DNA is first identified as containing 5-hmC in the purified genomic DNA. In one embodiment, the DNA that contains 5-hmC can be enriched and then further processed to identify specific genes in the sample that contain a significant amount of 5-hmC. In this way, reduction in the amounts of 5-hmC or the absence of 5-hmC (e.g., in genes that typically contain 5-hmC, as by comparison to a control sample) in a given sample can be identified. In one embodiment, measurement and quantitation is by detection of the 5-hmC in specific genes of the genomic DNA in the sample. In one embodiment, the threshold level of reduction of 5-hmC in a lesion is a ≧5 fold reduction in 5-hmC of the overall genes, or of specific genes, of the genomic DNA. Such a level of reduction indicates the melanocytic lesion is a melanoma.

Measurement and quantitation of 5-hmC in a sample can be achieved by measuring the 5-hmC that is labeled by an anti-5-hmC antibody-based detection system. Examples of such detections systems are dot blot assays and 5-hmC glucosylation assays (e.g., a T4 phage β-glucosyltransferase-mediated 5-hmC glucosylation assay). Other such assays are known in the art.

Experiments reported herein have further identified the cellular factors IDH2 and TET family genes (TET1, TET2 and TET3) as directly responsible for the loss of 5-hmC in melanomas. IDH2 and all three TET genes was shown to be significantly downregulated in melanomas. This finding indicates that knowledge of the expression levels of these genes in a tumor sample can be used for diagnosis of that tumor (e.g., melanocytic lesion). Another aspect of the invention arises from these findings and relates to a method of diagnosing a subject having a tumor (e.g., a melanocytic lesion). The method comprises determination of the level of expression of one or more of IDH2, TET1, TET2 and TET3 in a sample from the lesion and comparing that level of expression to that of an appropriate control. Identification of a threshold level of reduction of expression of the one or more genes in the sample is indicative of malignancy of the tumor from which the sample was obtained. Typically, the tissue sample is processed to thereby label the expression product of one or more of the genes (IDH2, TET1, TET2, TET3). The amount of the expression product of the one or more genes in the sample is measured by detection of the labeled expression product in the sample. The amount of labeled expression product(s) in the sample is quantitated (e.g., as compared to a healthy control) to thereby detect a threshold level of reduction of the expression product(s) in the sample. The diagnosis of a malignancy is made if a level of reduction of TET3 and/or IDH2 of >50% is detected, and/or if a level of reduction of TET1 and/or TET2 of >75% is detected.

Expression products targeted for detection of expression may be mRNA encoding the protein product, the actual protein product, or enzymatic activity of the protein product (e.g., by detection of some output produced by the protein product). In one embodiment, laser capture microdissection of the tumor is performed and the resulting sample is then processed to quantitatively detect the nucleic acid or protein products (e.g., by PCR, or an antibody based detection system).

### Treatment Methods

The diagnostic and prognostic methods described herein may further include steps for treatment of the malignancy. Various treatments for malignancies, such as melanoma, are known in the art. Any such treatment or combination of such methods may be used.

Traditional therapy of melanoma involves a number of treatment options. These generally include surgery, chemotherapy, radiation therapy and immunotherapy (IL-2, other). In the case of surgery, treatment can vary and can include local excision, wide local excision, lymphadenectomy, sentinel lymph node biopsy and skin grafting. In the case of chemotherapy, a standard chemotherapeutic agent dacarbazine (DTIC) is administered to the patient in order to treat the cancer, generally through cancer cell death. In the case of radiation therapy, radiation is used as a palliative rather than a cure for melanoma. Radiation relieves bone pain and other symptoms caused by metastases to the bones, brain, and organs such as the liver. Although not curative, radiation treatment is being investigated for more widespread use in controlling other symptoms of skin cancer. In the case of immunotherapy (biologic treatment), a patient's natural immune system is raised or other immune compositions (IL-2) are administered to the patient against the cancer. In one embodiment, combination therapy is used.

The term “coadministration” or “combination therapy” is used to describe a therapy in which at least two active compounds in effective amounts are used to treat melanoma, including metastatic melanoma as otherwise described herein at the same time. Although the term coadministration preferably includes the administration of two active compounds to the patient at the same time, it is not necessary that the compounds be administered-to-the-patient at the same time, although effective amounts of the individual compounds will be present in the patient at the same time. Chemotherapeutic agents include without limitation, dacarbazine (DTIC), immunotherapeutic agents include, without limitation IL-2 and/or alpha-interferon.

The experimental results presented herein indicate that the restoration of activity of IDH2 and/or TET2 in a malignant cell sufficient to increase the 5-hmC in the genome of the cell will reduce the malignancy of the cell. This finding indicates that treatment can be achieved by delivery of an effective amount of an agent that increases the expression of IDH2 and/or TET2 to a malignant cell (e.g., a melanoma). An effective amount of the delivered agent is one that is sufficient to increase the 5-hmC in the genome of the cell. As such, one aspect of the invention relates to a method of decreasing the malignancy of a tumor cell (e.g., a melanoma) by contacting the cell with an effective amount of an agent that increases expression of IDH2 and/or TET2. The agent can increase the endogenous expression or be an agent that encodes IDH2 and/or TET2 for expression of the exogenous gene (e.g., on an expression vector). Alternatively, a functional protein or fragment of exogenous IDH2 or TET2 can be delivered to the cell under conditions appropriate for uptake and function of the delivered protein by the cell. A functional protein as the term is used herein refers to a catalytically active IDH2 or TET family enzyme, a functional IDH2 or TET family derivative, or a IDH2 or TET catalytically active fragment thereof.

In one embodiment, the nucleic acid sequences of one or more of IDH2 or TET2 are delivered using a viral vector or a plasmid. The viral vector can be, for example, a retroviral vector, a lentiviral vector or an adenoviral vector. In some embodiments, the viral vector is a non-integrating viral vector. In one embodiment, reprogramming is achieved by introducing more than one non-integrating vector (e.g., 2, 3, 4, or more vectors) to a cell, wherein each vector comprises a nucleic acid sequence encoding a different agent. In an alternate embodiment, more than one agent is encoded by a non-integrating vector and expression of the agent is controlled using a single promoter, polycistronic promoters, or multiple promoters. Non-viral approaches to the introduction of nucleic acids known to those skilled in the art can also be used with the methods described herein. Alternatively, activation of the endogenous genes encoding such transcription factors can be used.

Delivery to the cell may be by administration of the agent to the subject with the malignancy, in the form of a pharmaceutical composition comprising the agent. Administration is by means to contact the cell with an effective amount of the agent. Examples of such administration are provided herein.

### Prognosis

Aspects of the invention relate to determining the prognosis of a subject with a tumor or lesion by determination of the 5-hmC present in the tumor or lesion. The experimental results reported herein indicate that the quantitative levels of 5-hmC present in a tumor or lesion correlate to the staging parameter of the lesion. These staging parameters are known in the art to correlate with prognosis of a subject. As such, the 5-hmC levels in a tumor can be used to predict the likelihood of survival and/or recovery of the subject by correlating the detected level of reduction in the tumor by the methods described herein, with one or more melanoma staging parameters, and determining the prognosis of the subject based on that of the determined particulars of the staging parameter. Such staging parameters include Breslow depth, mitotic rate (mitosis), the presence or absence of ulceration, and actual graded stage of the melanoma. The prognostic indication of a melanoma by virtue of these staging parameters is well known in the art and discussed herein only by way of a summary.

The herein described methods for prognosis can be performed at initial diagnosis. The methods can also be performed throughout treatment as a method of monitoring treatment effectiveness. Methods of prognosis can further be performed during remission, and recurrence.

A tumor within the skin such as a melanoma can be measured by a number of quantitative systems. The vertical growth phase is felt to delineate the ability of a tumor (e.g., melanoma) to metastasize. During vertical growth, prognosis can be predicted by a number of measurements which include depth measurements, mitotic counts, and ulceration (Crowson A N, et al. Prognosticators of melanoma, the melanoma report, and the sentinel lymph node. Mod Pathol 2006 February; 19 Suppl 2: S71-87). The most useful prognostic indicators of primary cutaneous melanomas are Breslow depth and presence or absence of ulceration (Fecher L A, et al. Toward a molecular classification of melanoma. J Clin Oncol 2007 Apr. 20; 25(12): 1606-20; Balch C M, Soong S J, Atkins M B, et al. An evidence-based staging system for cutaneous melanoma. CA Cancer J Clin 2004 May-June; 54(3): 131-49). Breslow Depth quantifies the top-to-bottom measurement of the primary melanoma tumor in millimeters, wherein risk increases with thickness.

The following stages are identified in the progression of the melanoma disease state. Melanoma progresses from an early stage (in situ) through an invasive stage, a high risk melanoma stage, a regional metastatic stage and a distant metastatic stage with varying degrees of survivability, as set forth below:

### Melanoma Stage, Staging Factors and Prognosis

### Stage 0: Melanoma in Situ (Clark Level I), 99.9% Survival

### Stage I/II: Invasive Melanoma, 85-95% Survival

T1a: Less than 1.00 mm primary, w/o Ulceration, Clark Level II-III
T1b: Less than 1.00 mm primary, w/ Ulceration or Clark Level IV-V
T2a: 1.00-2.00 mm primary, w/o Ulceration

### Stage II: High Risk Melanoma, 40-85% Survival

T2b: 1.00-2.00 mm primary, w/ Ulceration
T3a: 2.00-4.00 mm primary, w/o Ulceration
T3b: 2.00-4.00 mm primary, w/ Ulceration
T4a: 4.00 mm or greater primary w/o Ulceration
T4b: 4.00 mm or greater primary w/ Ulceration

### Stage III: Regional Metastasis, 25-60% Survival

N1: Single Positive Lymph Node
N2: 2-3 Positive Lymph Nodes OR Regional Skin/In-Transit Metastasis
N3: 4 Positive Lymph Nodes OR Lymph Node and Regional Skin/In Transit Metastases

### Stage IV: Distant Metastasis, 9-15% Survival

M1a: Distant Skin Metastasis, Normal LDH
M1b: Lung Metastasis, Normal LDH
M1c: Other Distant Metastasis OR Any Distant Metastasis with Elevated LDH

The survival rates are based upon AJCC 5-year survival with proper treatment. The prognosis for early stages of melanoma is good as it can be treated successfully with early diagnosis. The prognosis for patients diagnosed with metastatic melanoma is poor, with survival rates of six to nine months. In one embodiment, a threshold level of reduction of 5-hmC is set at an amount equivalent to a staining score of 1. Detection of an amount of reduction equivalent to a staining score ≧1 indicates a good or high prognosis. Detection of an amount of reduction equivalent to a staining score <1 indicates a bad or low prognosis.

### Samples

Samples are typically derived directly from the tumor or lesion and comprise a representative portion of the lesion. Such samples are obtained typically by biopsy. The specific method of biopsy will depend upon the tumor type and location and can be determined by the skilled practitioner. The sample for use in detecting the 5-hmC can be from various tissues including but are not limited to tissue biopsy, tissue section, formalin fixed paraffin embedded (FFPE) specimens, nasal swab or nasal aspirate, bronchoalveolar lavage, breast aspirate, pleural effusion, peritoneal fluid, glandular fluid, amniotic fluid, cervical swab or vaginal fluid, ejaculate, semen, prostate fluid, conjunctival fluid, duodenal juice, pancreatic juice, bile, and stool. The samples are processed according to the intended labeling and detection method.

### Processing Samples to Label 5-hmC

The amount of 5-hmC in a tumor or lesion can be determined by processing a sample of the tumor or lesion to thereby label the 5-hmC present in the sample and then detecting the label. The specific method of processing will depend upon the desired method of labeling and detection of the 5-hmC. In one embodiment, the sample is cut into sections and formalin-fixed (e.g., for immunohistochemical or immunofluorescent analysis). Such samples can be processed for long term storage (e.g., paraffin embedded) or further processed upon preparation. Such samples may be further processed, such as by immunohistochemical analysis, immunofluorescence or laser capture microdissection. In one embodiment, the sample is processed to further purify the DNA prior to labeling. Examples of various types of processing are provided herein or otherwise known in the art.

Labeling of the 5-hmC in the sample can be achieved by a variety of methods. In one embodiment, the labeling utilizes an antibody that specifically recognizes 5-hmC. Such antibodies are available commercially. In one embodiment, the antibody has a detectable label attached. In one embodiment, the antibody is used to separate the 5-hmC (e.g., DNA) from the non-5-hmC DNA when in solution. This separation can then be followed by sequencing of the genes that have the 5-hmC.

### Detection of 5-hmC

The content of 5-hmC in the sample is determined by comparing the content of 5-hmC from a sample with reference content measured from a healthy sample obtained from the same subject, or a healthy or cancer-free individual. 5-hmC can be detected by many methods well known in the art. A variety of chromatography-based technologies including, without limitation, capillary electrophoresis (CE), mass spectrometry (MS) HPLC and thin layer chromatography (TLC) (Kriaucionis S et al, Science. 2009 May 15; 324(5929):929-30; Penn N W et al, Biochem. J. (1972) 126 (781-790); Szwagierczak A et al, Nucleic Acids Res. 2010 October; 38(19):e181) can be used for measuring 5-hmC or the biomolecules having general structure of 5-hmC. In one embodiment, an immunoassay method specifically developed for quantification of 5-hmC such as that described in U.S. Patent Publication 20120003663 can be used.

Preferably immunoassay techniques including competitive and non-competitive immunoassays can be used. A variety of immunoassay techniques include, but are not limited to, enzyme immunoassays (EIA) such as enzyme multiplied immunoassay technique (EMIT), enzyme-linked immunosorbent assay (ELISA), enzyme-linked immunosorbent spot (ELISPOT), microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with time-resolved fluorescence (TRF) assay such as DEFLIA assays; luminescent oxygen channeling assay (LOCI) such as A1phaLISA or AlphaScreen assays; laser induced fluorescence; liposome immunoassays; and immunosensors. Other immunoassay methods such as dot blot immunoassay, immunohistochemical staining, and immunofluorescence assays can be also used. Immunoassay methods and protocols are generally described in the prior art.

Embodiments of the herein described methods for rapid analysis of large numbers of samples are envisioned. For example, samples can be processed and/or measurements can be made in multi-well plate, microchip, microscope slide, and nitrocellulose membranes. Such embodiments may involve one or more automated steps performed by a non-human machine, examples of which are described below.

### IDH2 and TET Family Proteins

Isocitratedehydrogenases (IDHs) are metabolic enzymes in the TCA cycle and catalyze the oxidative decarboxylation of isocitrate to α-ketoglutarate (α-KG). IDHs can be classified into two groups (depending on the types of e-acceptor): (1) NAD+-dependent isocitratedehydrogenases, such as IDH3A, IDH3B, IDH3G, which form heterotetramer α2βγ, play an irreversible step of TCA cycle, and are found in the mitochondrial matrix; and (2) NDAP+-dependent isocitratedehydrogenases, such as IDH1, IDH2, which form homodimers, are involved in NADPH regeneration for anabolic pathways, and can be found in the mitochondrial matrix (IDH2) or cytoplasm/peroxisome (IDH1). The nucleic acid sequence of human IDH2 and the encoded protein sequence are deposited under NCBI Reference Sequence: NM_{—}002168.2.

The TET family of proteins comprises the nucleotide sequences of TET1, TET2, and TET3, with GenBank nucleotide sequence IDs: GeneID: NM_{—}030625.2 (TET1), GeneID: NM_{—}001127208.1 (TET2), GeneID: NM_{—}144993.1 (TET3), and the protein sequences of TET1, TET2, and TET3 with GenBank peptide sequence IDs: NP_{—}085128 (TET1), NP_{—}001120680 (TET2), and TET3 GenBank Peptide ID: NP_{—}659430.

### Types of Cancer

The present invention is envisioned for use in diagnosis, prognosis and treatment of a solid tumor. Examples of solid tumors, such as sarcomas and carcinomas, include without limitation, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma), melanoma, esophageal cancer, liver cancer, gastrointestinal cancer, colon cancer or a lung carcinoma.

The methods described herein can be applied, for example, to skin cancer. Skin cancer is a malignant growth on the skin which can have many causes Skin cancer generally develops in the epidermis (the outermost layer of skin), so a tumor is usually clearly visible. This makes most nonmelanoma skin cancers detectable in the early stages Skin cancer represents the most commonly diagnosed malignancy, surpassing lung, breast, colorectal and prostate cancer.

The most common type of skin cancer is nonmelanoma skin cancer. Nonmelanoma skin cancers include all skin cancers except malignant melanoma (cancer that develops from melanocytes, the pigment-producing cells of the skin) There are many types of nonmelanoma skin cancers. Two common types of nonmelanoma skin cancer are basal cell carcinoma and squamous cell carcinoma. These two types of skin cancer are also known as keratinocyte carcinomas.

Basal cell carcinoma begins in the lowest layer of the epidermis, called the basal cell layer. About 70% to 80% of all skin cancers in men and 80% to 90% in women are basal cell carcinomas. They usually develop on sun-exposed areas, especially the head and neck. Basal cell carcinoma is slow growing. It is highly unusual for a basal cell cancer to spread to lymph nodes or to distant parts of the body. However, if a basal cell cancer is left untreated, it can grow into nearby areas and invade the bone or other tissues beneath the skin. After treatment, basal cell carcinoma can recur in the same place on the skin. Also, new basal cell cancers can start elsewhere on the skin. Within 5 years of being diagnosed with one basal cell cancer, 35% to 50% of people develop a new skin cancer.

Squamous cell carcinomas account for about 10% to 30% of all skin cancers. They commonly appear on sun-exposed areas of the body such as the face, ear, neck, lip, and back of the hands. Squamous cell carcinomas can also develop in scars or skin ulcers elsewhere. These carcinomas are generally more aggressive than basal cell cancers. Squamous cell carcinomas can sometimes start in actinic keratoses. Squamous cell carcinoma in situ (also called Bowen disease) is the earliest form of squamous cell skin cancer and involves cells that are within the epidermis and have not invaded the dermis.

Less common types of nonmelanoma skin cancer include Kaposi sarcoma, cutaneous lymphoma, skin adnexal tumors and various types of sarcomas and Merkel cell carcinoma. Together, these types of nonmelanoma skin cancer account for less than 1% of nonmelanoma skin cancers.

### Isolation of DNA

In one embodiment, the obtained sample is processed to isolate the genomic DNA contained therein. DNA can be isolated by lysis of cells with lysis buffer containing a sodium salt, tris-HCl, EDTA, and detergents such as sodium dodecyl sulphate (SDS) or cetyltrimethylammonium bromide (CATB). Tissue fragments should be homogenized before lysing. For example, disaggregating of tissue fragments can be performed by stroking 10-50 times, depending on tissue type, with a Dounce homogenizer. DNA can be further purified by mixing with a high concentration of sodium chloride and then adding into a column pre-inserted with a silica gel, a silica membrane, or a silica filter. The DNA that binds to the silica matrix is washed by adding a washing buffer and eluted with TE buffer or water. DNA can also be isolated and purified by using commercially available DNA extraction kits such as QiaAmp tissue kits.

Body fluid should be pre-treated under appropriate condition prior to DNA extraction. Cells obtained from biological fluid samples can be collected by the procedures described in prior art. For example, collection of cells in a urine sample can simply be achieved by simply centrifugation, while collection of cells in a sputum sample requires DTT treatment of sputum followed by filtering through a nylon gauze mesh filter and then centrifugation. If a stool sample is used, a stool stabilizing and homogenizing reagents should be added to stabilize DNA and remove stool particles. Human DNA fraction from total stool DNA then can be primarily isolated or purified using commercially available stool DNA isolation kits such as Qiagen DNA Stool Mini Kit (using the protocol for human DNA extraction) or be captured by methyl-binding domain (MBD)-based methylated DNA capture methods after total DNA isolation.

### Automation

It is to be understood that one or more of the steps in the methods described herein can be performed by a non-human machine. For example, measuring the amount of 5-hmC in a sample can be performed by detection of labeled 5-hmC by a non-human machine. Quantitation of the amount of 5-hmC in the sample that is detected can be compared to a healthy control or predetermined standard level, by a non-human machine, to thereby generate an output of the reading. In such an embodiment, the non-human machine can be adjusted to produce a positive readout upon detection of the threshold level of reduction that is input into the detection and quantitation system. The identification of these specific threshold levels is made possible by the herein described findings. The knowledge of these levels allows for automation with rapid, reliable and reproducible output by such a detection system.

### Systems for Performance of the Methods

Embodiments of the invention described herein also relate to systems (and computer readable medium for causing computer systems) to perform a method for determining whether an individual has a specific disease or disorder, a pre-disposition for a specific disease or disorder, and also for prognosis of an individual with a specific disease or disorder based on expression profiles and 5-hmC landscape of tumor tissue of the subject described herein.

Embodiments of the invention have been described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, or computer readable medium, may define instructions, for example, as part of one or more programs, that, as a result of being executed by a computer, instruct the computer to perform one or more of the functions described herein (e.g., in relation to system, or computer readable medium), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system10, or computer readable medium described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module, a storage device, a comparison module and a display module. The functional modules can be executed on one, or multiple, computers, or by using one, or multiple, computer networks. The determination module has computer executable instructions to provide sequence information in computer readable form. As used herein, “sequence information” refers to any nucleotide and/or amino acid sequence, including but not limited to full-length nucleotide and/or amino acid sequences, partial nucleotide and/or amino acid sequences, or mutated sequences. Sequence information also refers to the 5-hmC in the DNA of a sample, and to the amount or concentration of the 5-hmC. Moreover, information “related to” the sequence information includes detection of the presence or absence of a sequence (e.g., detection of a mutation or deletion), determination of the concentration of a sequence in the sample (e.g., amino acid sequence expression levels, or nucleotide (RNA or DNA) expression levels), and the like. The term “sequence information” is intended to include the presence or absence of post-translational modifications (e.g. phosphorylation, glycosylation, summylation, farnesylation, and the like).

As an example, determination modules for determining sequence information may include known systems for automated sequence analysis including but not limited to Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics FluorImager™ 575, SI Fluorescent Scanners, and Molecular Dynamics FluorImager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England).

Alternative methods for determining sequence information, i.e. determination modules, include systems for protein and DNA analysis. For example, mass spectrometry systems including Matrix Assisted Laser Desorption Ionization—Time of Flight (MALDI-TOF) systems and SELDI-TOF-MS ProteinChip array profiling systems; systems for analyzing gene expression data (see, for example, published U.S. patent application, Pub. No. U.S. 2003/0194711); systems for array based expression analysis: e.g., HT array systems and cartridge array systems such as GeneChip® AutoLoader, Complete GeneChip® Instrument System, GeneChip® Fluidics Station 450, GeneChip® Hybridization Oven 645, GeneChip® QC Toolbox Software Kit, GeneChip® Scanner 3000 7G plus Targeted Genotyping System, GeneChip® Scanner 3000 7G Whole-Genome Association System, GeneTitan™ Instrument, and GeneChip® Array Station (each available from Affymetrix, Santa Clara, Calif.); automated ELISA systems (e.g., DSX® or DK® (available from Dynax, Chantilly, Va.) or the Triturus® (available from Grifols USA, Los Angeles, Calif.), The Mago® Plus (available from Diamedix Corporation, Miami, Fla.); Densitometers (e.g. X-Rite-508-Spectro Densitometer® (available from RP Imaging™, Tucson, Ariz.), The HYRYS™ 2 HIT densitometer (available from Sebia Electrophoresis, Norcross, Ga.); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

The sequence information determined in the determination module can be read by the storage device. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon sequence information or expression level information. Such information may be provided in digital form that can be transmitted and read electronically, e.g., via the Internet, on diskette, via USB (universal serial bus) or via any other suitable mode of communication.

As used herein, “expression level information” refers to any nucleotide and/or amino acid expression level information, including but not limited to full-length nucleotide and/or amino acid sequences, partial nucleotide and/or amino acid sequences, or mutated sequences. Moreover, information “related to” the expression level information includes detection of the presence or absence of a sequence (e.g., presence or absence of an amino acid sequence, nucleotide sequence, or post translational modification), determination of the concentration of a sequence in the sample (e.g., amino acid sequence levels, or nucleotide (RNA or DNA) expression levels, or level of post translational modification), and the like.

As used herein, “stored” refers to a process for encoding information on the storage device. Those skilled in the art can readily adopt any of the presently known methods for recording information on known media to generate manufactures comprising the sequence information or expression level information.

A variety of software programs and formats can be used to store the sequence information or expression level information on the storage device. Any number of data processor structuring formats (e.g., text file or database) can be employed to obtain or create a medium having recorded thereon the sequence information or expression level information.

By providing sequence information or expression level information in computer-readable form, one can use the sequence information or expression level information in readable form in the module to compare a specific sequence or expression profile with the reference data within the storage device 30. For example, search programs can be used to identify fragments or regions of the sequences that match a particular sequence (reference data, e.g., sequence information obtained from a control sample) or direct comparison of the determined expression level can be compared to the reference data expression level (e.g., sequence information obtained from a control sample). The comparison made in computer-readable form provides a computer readable comparison result which can be processed by a variety of means. Content based on the comparison result can be retrieved from the module to indicate a specific disease or disorder or prognosis of an individual with a specific disease or disorder as described herein.

In one embodiment the reference data stored in the storage device 30 to be read by the module is sequence information data obtained from a control biological sample of the same type as the biological sample to be tested. Alternatively, the reference data are a database, e.g., a part of the entire genome sequence of an organism, or a protein family of sequences, or an expression level profile (RNA, protein or peptide). In one embodiment the reference data are sequence information or expression level profiles that are indicative of a specific disease or disorder or prognosis of an individual with a specific disease or disorder as described herein.

In one embodiment, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting information can be stored in a relational data base that may be employed to determine homologies between the reference data or genes or proteins within and among genomes.

The “comparison module” can use a variety of available software programs and formats for the comparison operative to compare sequence information determined in the determination module to reference data. In one embodiment, the module is configured to use pattern recognition techniques to compare sequence information from one or more entries to one or more reference data patterns. The module may be configured using existing commercially-available or freely-available software for comparing patterns, and may be optimized for particular data comparisons that are conducted. The module provides computer readable information related to the sequence information that can include, for example, detection of the presence or absence of a sequence (e.g., detection of a mutation or deletion (protein or DNA), information regarding distinct alleles, detection of post-translational modification, or omission or repetition of sequences); determination of the concentration of a sequence in the sample (e.g., amino acid sequence/protein expression levels, or nucleotide (RNA or DNA) expression levels, or levels of post-translational modification), and detection of the presence or absence of 5-hmC in one or more genes, quantitative detection of the presence of 5-hmC in a biological sample, or across the genomic landscape of the biological sample, or determination of an expression profile.

In one embodiment, the module permits the prediction of protein sequences from polynucleotide sequences, permits prediction of open reading frames (ORF), or permits prediction of homologous sequence information in comparison to reference data, i.e., homologous protein domains, homologous DNA or RNA sequences, or homologous exons and/or introns.

In one embodiment, the module uses sequence information alignment programs such as BLAST (Basic Local Alignment Search Tool) or FAST (using the Smith-Waternan algorithm) may be employed individually or in combination. These algorithms determine the alignment between similar regions of sequences and a percent identity between sequences. For example, alignment may be calculated by matching, bases-by-base or amino acid-by amino-acid.

The module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the module performs comparisons with mass-spectometry spectra to detect 5-hmC present in a sample (e.g., a whole sample or purified genomic DNA).

In one embodiment, the module performs comparisons with mass-spectometry spectra to detect polypeptides in a sample. For example, comparisons of peptide fragment sequence information can be carried out using spectra processed in MATLB with script called “Qcealign” (see for example WO2007/022248, herein incorporated by reference) and “Qpeaks” (Spectrum Square Associates, Ithaca, N.Y.), or Ciphergen Peaks 2.1™ software. The processed spectra can then be aligned using alignment algorithms that align sample data to the control data using minimum entropy algorithm by taking baseline corrected data (see for example WIPO Publication WO2007/022248, herein incorporated by reference). The comparison result can be further processed by calculating ratios. Protein expression profiles can be discerned.

In one embodiment, computational algorithms are used in the module, such as expectation-maximization (EM), subtraction and PHASE are used in methods for statistical estimation of haplotypes (see, e.g., Clark, A. G. Mol Biol Evol 7:111-22 (1990); Stephens, M., Smith, N.J. & Donnelly, P. Am J Hum Genet 68:978-89 (2001); Templeton, A. R., Sing, C. F., Kessling, A. & Humphries, Genetics 120:1145-54 (1988)).

Various algorithms are available which are useful for comparing data and identifying the predictive gene signatures. For example, algorithms such as those identified in Xu et al., Physiol. Genomics 11:11-20 (2002). There are numerous software available for detection of SNPs and polymorphisms that can be used in the comparison module, including, but not limited to: HaploSNPer, a web-based program for detecting SNPs and alleles in user-specified input sequences from both diploid and polyploid species (available on the world-wide web at bioinformatics.nl/tools/haplosnper/; see also Tang et al., BMC Genetics 9:23 (2008)); Polybayes, a tool for SNP discovery in redundant DNA sequences (Marth, G T., et al., Nature Genetics 23(4):452-6 (1999); SSAHA-SNP, a polymorphism detection tool that uses the SSAHA alignment algorithm (available from Wellcome Trust Sanger Institute, Cambridge, United Kingdom, see also Ning Z., et al., Genome Research 11(10):1725-9 (2001)); Polyphred, A SNP discovery package built on phred, phrap, and consed tools (available on the world-wide web, see Nickerson, D A et al., Nucleic Acids Research 25(14):2745-51 (1997)); NovoSNP, a graphical Java-based program (PC/Mac/Linux) to identify SNPs and indels (available on the world-wide web, see Weckx, S. et al., Genome Research 15(3):436-442 (2005)); SNPdetector™, for automated identification of SNPs and mutations in fluorescence-based resequencing reads (available from Affymetrix, Santa Clara, Calif.), see also Zhang et al. PLoS Comput Biol (5):e53 (2005). SNPdetector runs on Unix/Linux platform and is available publicly; Affymetrix (Santa Clara, Calif.) has multiple data analysis software that can be used, for example Genotyping Console™ Software, GeneChip® Sequence Analysis Software (GSEQ), GeneChip® Targeted Genotyping Analysis Software (GTGS) and Expression Console™ Software.

In one embodiment, the module compares gene expression profiles. For example, detection of gene expression profiles can be determined using Affymetrix Microarray Suite software version 5.0 (MAS 5.0) (available from Affymetrix, Santa Clara, Calif.) to analyze the relative abundance of a gene or genes on the basis of the intensity of the signal from probe sets, and the MAS 5.0 data files can be transferred into a database and analyzed with Microsoft Excel and GeneSpring 6.0 software (available from Agilent Technologies, Santa Clara, Calif.). The detection algorithm of MAS 5.0 software can be used to obtain a comprehensive overview of how many transcripts are detected in given samples and allows a comparative analysis of 2 or more microarray data sets.

In one embodiment, the module compares protein expression profiles. Any available comparison software can be used, including but not limited to, the Ciphergen Express (CE) and Biomarker Patterns Software (BPS) package (available from Ciphergen Biosystems, Inc., Freemont, Calif.). Comparative analysis can be done with protein chip system software (e.g., The Proteinchip Suite (available from Bio-Rad Laboratories, Hercules, Calif.). Algorithms for identifying expression profiles can include the use of optimization algorithms such as the mean variance algorithm (e.g. JMP Genomics algorithm available from JMP Software Cary, N.C.).

In one embodiment of the invention, pattern comparison software is used to determine whether patterns of expression, mutations, or 5-hmC content in a sample are indicative of a disease.

The module provides computer readable comparison result that can be processed in computer readable form by predefined criteria, or criteria defined by a user, to provide a content based in part on the comparison result that may be stored and output as requested by a user using a display module. The display module enables display of a content based in part on the comparison result for the user, wherein the content is a signal indicative of the specific disease or disorder or prognosis of a subject with the specific disease or disorder. Such signal, can be for example, a display of content indicative of the presence or absence of the specific disease or disorder or prognosis of a subject with the specific disease or disorder on a computer monitor, a printed page of content indicating the presence or absence of the specific disease or disorder or prognosis of a subject with the specific disease or disorder from a printer, or a light or sound indicative of the presence or absence of the specific disease or disorder or prognosis of a subject with the specific disease or disorder.

The content based on the comparison result may include an expression profile of one or more proteins, or an expression profile of one or more genes. In one embodiment, the content based on the comparison includes a sequence of a particular gene or protein and a determination of the presence of one or more mutations, or specific post-translational modification. In one embodiment, the content based on the comparison result is merely a signal indicative of the presence or absence of the specific disease or disorder or prognosis of a subject with the specific disease or disorder.

In one embodiment of the invention, the content based on the comparison result is displayed a on a computer monitor. In one embodiment of the invention, the content based on the comparison result is displayed through printable media. In one embodiment of the invention, the content based on the comparison result is displayed as an indicator light or sound. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the sequence information, e.g., display of an indication of the presence or absence of mutation or deletion (DNA or protein); display of expression levels of an amino acid sequence (protein); display of nucleotide (RNA or DNA) expression levels; display of expression, SNP, or mutation profiles, or haplotypes, or display of information based thereon. In one embodiment, the sequence information of the reference sample data is also displayed.

In one embodiment, the display module displays the comparison result and whether the comparison result is indicative of a disease, e.g., whether the profile of the 5-hmG and/or the expression profile of the particular proteins, or genes of IDH2, TET1, TET2 and TET3 is indicative of the malignancy in the subject or prognosis of the subject.

In one embodiment, the content based on the comparison result that is displayed is a signal (e.g. positive or negative signal) indicative of the presence or absence of the specific disease or prognosis of the subject with the disease, thus only a positive or negative indication may be displayed.

The present invention therefore provides for systems (and computer readable medium for causing computer systems) to perform methods for determining whether an individual has a specific disease or a pre-disposition, for a specific disease, or the prognosis of a subject with the specific disease, based on expression profiles or sequence information described herein.

System, and computer readable medium, are merely an illustrative embodiments of the invention for performing methods of determining whether an individual has a specific disease or disorder or a pre-disposition, for the specific disease or disorder, or the prognosis of a subject with the disease or disorder based on expression profiles or sequence information, and is not intended to limit the scope of the invention. Variations of system, and computer readable medium, are possible and are intended to fall within the scope of the invention.

The modules of the system or used in the computer readable medium, may assume numerous configurations. For example, function may be provided on a single machine or distributed over multiple machines.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.

1. A method of detecting malignancy in a tumor of a subject comprising,
a) processing a sample of the tumor to thereby label the 5-hmC present in the sample;
b) measuring the amount of 5-hmC in the sample by detection of the labeled 5-hmC in the sample;
c) quantitating the amount of 5-hmC in the sample as compared to a healthy control to thereby detect a threshold level of reduction of 5-hmC in the sample; and
d) characterizing the tumor as malignant when a threshold level of reduction of 5-hmC is detected.
   2. The method of paragraph 1, wherein the tumor is selected from the group consisting of a breast tumor, a colon tumor, skin tumor, ovarian tumor, lung tumor, liver tumor, prostate tumor, brain tumor, and kidney tumor.
   3. The method of paragraph 1 or 2, wherein the processing step a) is by immunohistochemical staining or by immunofluorescence.
   4. A method of diagnosing a subject with a melanocytic lesion comprising,
a) processing a tissue sample of the melanocytic lesion of the subject to thereby label the 5-hmC present in the tissue sample;
b) measuring the amount of 5-hmC in the sample by detection of the labeled 5-hmC in the tissue sample;
c) quantitating the amount of 5-hmC in the tissue sample as compared to a healthy control to thereby detect a threshold level of reduction of 5-hmC in the sample; and
d) characterizing the melanocytic lesion by the detected level of reduction of 5-hmC to thereby diagnose the subject.
   5. The method of paragraph 4, wherein the processing step a) is by immunohistochemical staining or by immunofluorescence.
   6. The method of paragraph 5, wherein quantitating step c) is by assignment of a 5-hmC staining score.
   7. The method of any one of paragraphs 4-6, wherein the threshold level of reduction is equivalent to:
a) a 5-hmC staining score of ≦2 which indicates the melanocytic lesion is a stage 2-3 melanoma, with a Breslow of >1 mm, and >1 mitosis;
b) a 5-hmC staining score of ≧3 which indicates the melanocytic lesion is a stage 1 melanoma, with a Breslow of ≦1 mm, and ≦1 mitosis;
c) a 5-hmC staining score of ≧1.5 which indicates the melanocytic lesion is a melanoma without ulceration; or
d) a 5-hmC staining score of <1.5 which indicates the melanocytic lesion is a melanoma with ulceration.
   8. The method of paragraph 2, wherein quantitating step c) is by assignment of a 5-hmC cell count score.
   9. The method of paragraph 4, 5, or 8 wherein the threshold level of reduction is equivalent to:
a) a 5-hmC cell count score of ≧2 which indicates the melanocytic lesion is a benign melanocytic nevus;
b) a 5-hmC cell count score of ≧3 which indicates the melanocytic lesion is a benign melanocytic nevus;
c) a 5-hmC cell count score of <1 which indicates the melanocytic lesion is a primary cutaneous melanoma or a visceral metastatsic melanoma;
d) a 5-hmC cell count score of <0.5 which indicates the melanocytic lesion is a primary cutaneous melanoma or a visceral metastatsic melanoma; or
e) a 5-hmC cell count score of <0.25 which indicates the melanocytic lesion is a lymphnode metastatic melanoma.
   10. The method of paragraph 4, wherein processing step a) is by purifying genomic DNA from a tissue sample of the melanocytic lesion and measuring step b) is by sequencing genomic DNA identified as containing 5-hmC in the purified genomic DNA.
   11. The method of paragraph 4, wherein processing step a) is by purifying genomic DNA from a tissue sample of the melanocytic lesion and measuring step b) is by detection of the 5-hmC in specific genes of the genomic DNA.
   12. The method of paragraph 10, wherein the threshold level of reduction is a ≧5 fold reduction in 5-hmC of the specific genes of the genomic DNA which indicates the melanocytic lesion is a melanoma.
   13. The method of paragraph 4, wherein processing step a) is by purifying genomic DNA from a tissue sample of the melanocytic lesion and measuring step b) is by an anti-5-hmC antibody-based detection system.
   14. The method of paragraph 13, wherein the anti-5-hmC antibody-based detection system is a dot blot assay.
   15. The method of paragraph 10, wherein the 5-hmC levels are detected by a 5-hmC glucosylation assay.
   16. The method of paragraph 15 wherein the 5-hmC glucosylation assay is a T4 phage β-glucosyltransferase-mediated 5-hmC glucosylation assay.
   17. A method of diagnosing a subject having a melanocytic lesion comprising,
a) processing a tissue sample of the melanocytic lesion of the subject to thereby label the expression product of one or more of the genes IDH2, TET1, TET2, TET3;
b) measuring the amount of the expression product of the one or more genes in the sample by detection of the labeled expression product in the tissue sample;
c) quantitating the amount of labeled expression product(s) in the sample as compared to a healthy control to thereby detect a threshold level of reduction of the expression product(s) in the sample; and
d) diagnosing the subject as having a malignancy if a level of reduction of TET3 and/or IDH2 of >50% is detected, and/or if a level of reduction of TET1 and/or TET2 of >75% is detected.
   18. A method of determining prognosis of a subject with a melanocytic lesion comprising,
a) processing a tissue sample of the melanocytic lesion of the subject to thereby label the 5-hmC present in the tissue sample;
b) measuring the amount of 5-hmC in the sample by detection of the labeled 5-hmC in the tissue sample;
c) quantitating the amount of 5-hmC in the tissue sample as compared to a healthy control to thereby detect a threshold level of reduction of 5-hmC in the sample; d) correlating the detected threshold level of reduction in step c) with one or more melanoma staging parameters; and
e) determining the prognosis of the subject based on that of the staging parameter to which the amount of 5-hmC correlates.
   19. The method of paragraph 18, wherein the staging parameter is selected from the group consisting of Breslow depth, mitosis rate, presence or absence of ulceration, overall stage of melanoma, melanocytic lesion type, and combinations thereof.
   20. The method of paragraph 18, wherein the processing step a) is by immunohistochemical staining or by immunofluorescence.
   21. The method of paragraph 20, wherein quantitating step c) is by assignment of a 5-hmC staining score.
   22. The method of any one of paragraphs 18-21, wherein the threshold level of reduction is equivalent to:
a) a 5-hmC staining score of ≦2 which indicates the melanocytic lesion is a stage 2-3 melanoma, with a Breslow of >1 mm, and >1 mitosis;
b) a 5-hmC staining score of ≧3 which indicates the melanocytic lesion is a stage 1 melanoma, with a Breslow of ≦1 mm, and ≦1 mitosis;
c) a 5-hmC staining score of ≧1.5 which indicates the melanocytic lesion is a melanoma without ulceration; or
d) a 5-hmC staining score of <1.5 which indicates the melanocytic lesion is a melanoma with ulceration.
   23. The method of paragraph 20, wherein quantitating step c) is by assignment of a 5-hmC cell count score.
   24. The method of any one of paragraphs 18-20 or 23, wherein the threshold level of reduction is equivalent to:
a) a 5-hmC cell count score of ≧2 which indicates the melanocytic lesion is a benign melanocytic nevus;
b) a 5-hmC cell count score of ≧3 which indicates the melanocytic lesion is a benign melanocytic nevus;
c) a 5-hmC cell count score of <1 which indicates the melanocytic lesion is a primary cutaneous melanoma or a visceral metastatsic melanoma;
d) a 5-hmC cell count score of <0.5 which indicates the melanocytic lesion is a primary cutaneous melanoma or a visceral metastatsic melanoma; or
e) a 5-hmC cell count score of <0.25 which indicates the melanocytic lesion is a lymphnode metastatic melanoma.
   25. A method for treating a subject with a melanoma comprising contacting melanoma cells of the subject with an effective amount of an agent that increases expression of IDH2 and/or TET2 sufficient to increase 5-hmC in the genome of the cell.
   26. The method of paragraph 25, wherein the agent is selected from an expression vector encoding IDH2 and/or TET2, a regulatory molecule which increases transcription or translation of the IDH2 and/or TET2 gene, and combinations thereof.
   27. The method of one of paragraphs 25 or 26, wherein contacting is by administering to the subject a therapeutic amount of a pharmaceutical composition comprising the agent.
   28. The method of paragraph 27, wherein administering is intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, or intratumor.

The invention is further illustrated by the following examples, which should not be construed as further limiting.

### Examples

Using melanoma as a paradigm of aggressive cancer, “loss-of-5-hmC” as a new epigenetic hallmark of melanoma is reported. The significant impact of 5-hmC, IDH2 and TET2 in melanoma progression is functionally characterized. Importantly, it is shown that the activity of IDH2 and TET2 enzymes required for the production of 5-hmC and the re-establishment of the 5-hmC landscape in melanoma cells is essential to regulation of melanoma virulence, contributing to the current understanding of cancer epigenetics.

### 5-hmC Level is High in Mature Melanocytes and Nevi and Lost in Human Melanomas

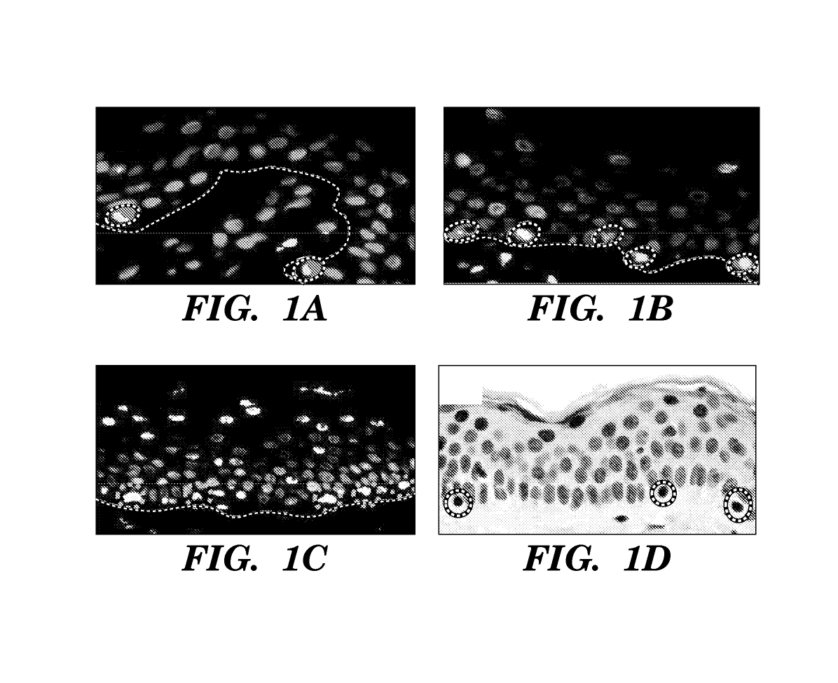

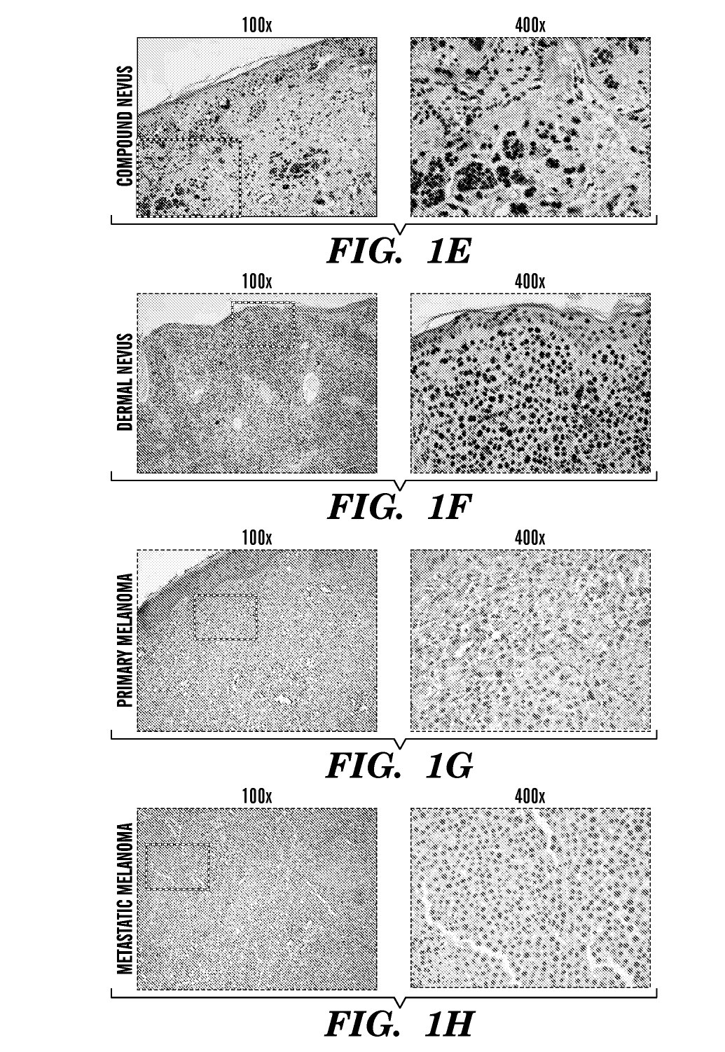

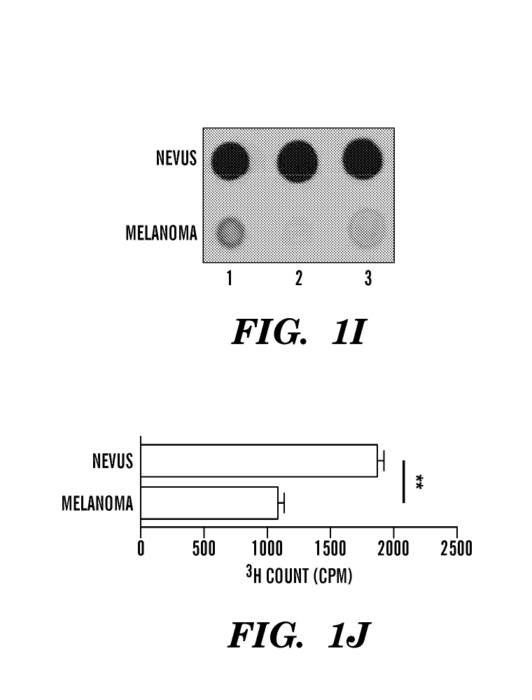

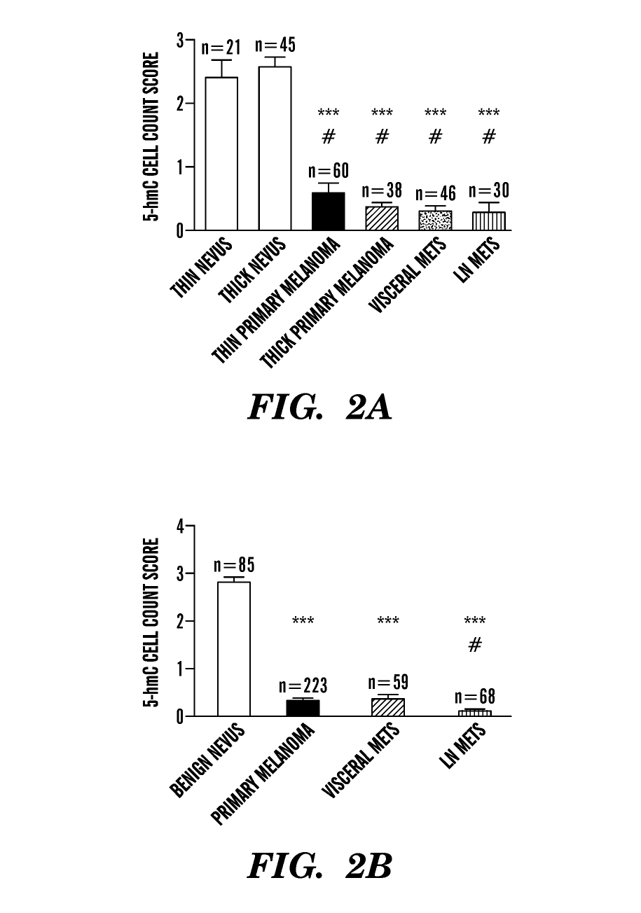

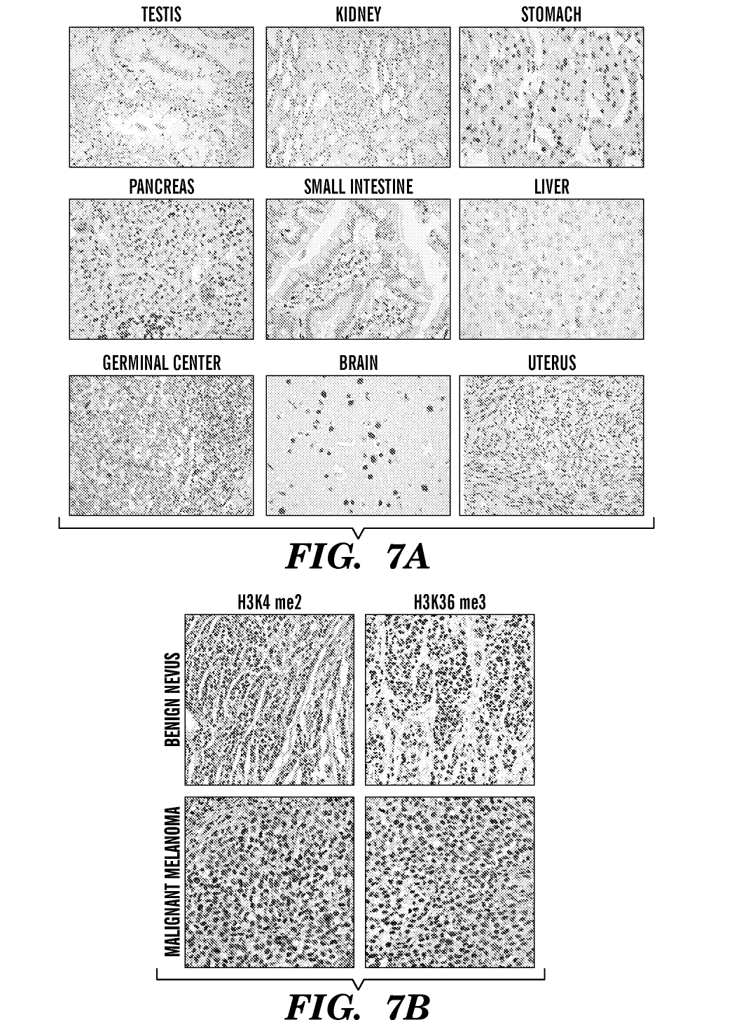

High levels of 5-hmC were detected by immunofluorescent (IF) staining in the nuclei of isolated melanocytes that co-expressed MART-1, a melanocyte specific marker, within the epidermal basal cell layer (FIGS. 1A and 1B). A more sensitive method for IF or immunohistochemical (IHC) staining of 5-hmC using formalin-fixed, paraffin-embedded tissue sections resulted in loss of the MART-1 epitope, but significantly improved the detection of 5-hmC as demonstrated by staining in normal human tissues (FIG. 7A). By this method, strong IF staining of 5-hmC was detected in melanocytes within the otherwise negative basal layer, as seen in FIGS. 1A and 1B, as well as variably within more differentiated suprabasal keratinocytes (FIG. 1C). The IF staining pattern was confirmed by IHC staining (FIG. 1D), and this more sensitive method for 5-hmC detection was utilized for all subsequent studies of melanocytic nevi and melanomas.

Over 50 individual cases of representative melanocytic lesions, including benign nevi, primary melanomas, and metastatic melanomas were initially evaluated. Benign nevus cases (n=30) showed strong nuclear 5-hmC staining, whereas virtually all tumor cells in primary (n=15) and metastatic (n=10) melanomas showed partial or complete loss of 5-hmC (FIGS. 1E-1H). Significant differences in other epimarks were not observed between benign nevi and melanomas (FIG. 7B and Table 1), suggesting the unique discriminatory nature of 5-hmC staining as it relates to cells of melanocytic lineage. Genomic DNA was then purified from nevi and melanomas and the observation that higher 5-hmC levels in nevi than in melanomas was confirmed by two independent methods, the anti-5-hmC antibody-based dot-blot (FIG. 1I) and T4 Phage β-glucosyltransferase-mediated 5-hmC glucosylation assay (FIG. 1J). Taken together, these data demonstrate that while a high level of 5-hmC is a distinctive epigenetic signature for benign melanocytes and nevi, significantly diminished or complete loss of 5-hmC is a feature of melanomas.

### 5-hmC is a Putative Molecular Marker of Melanoma Progression

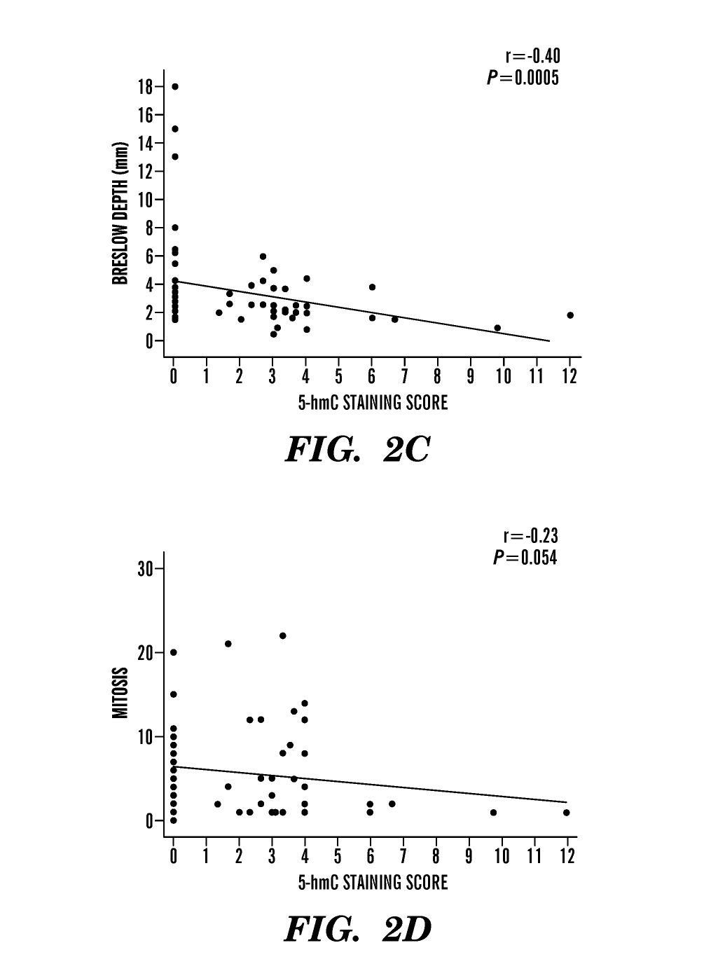

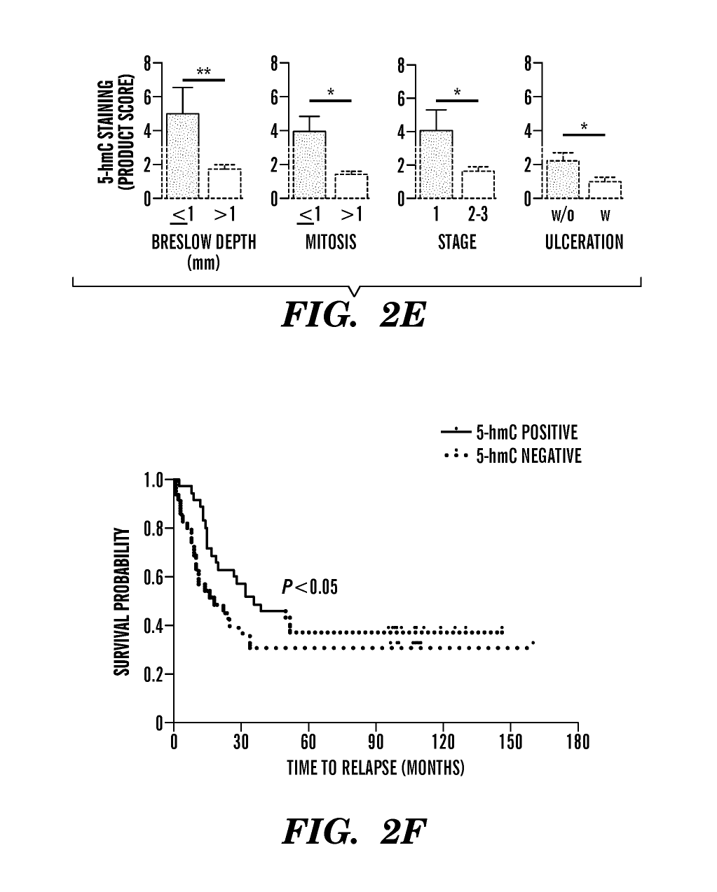

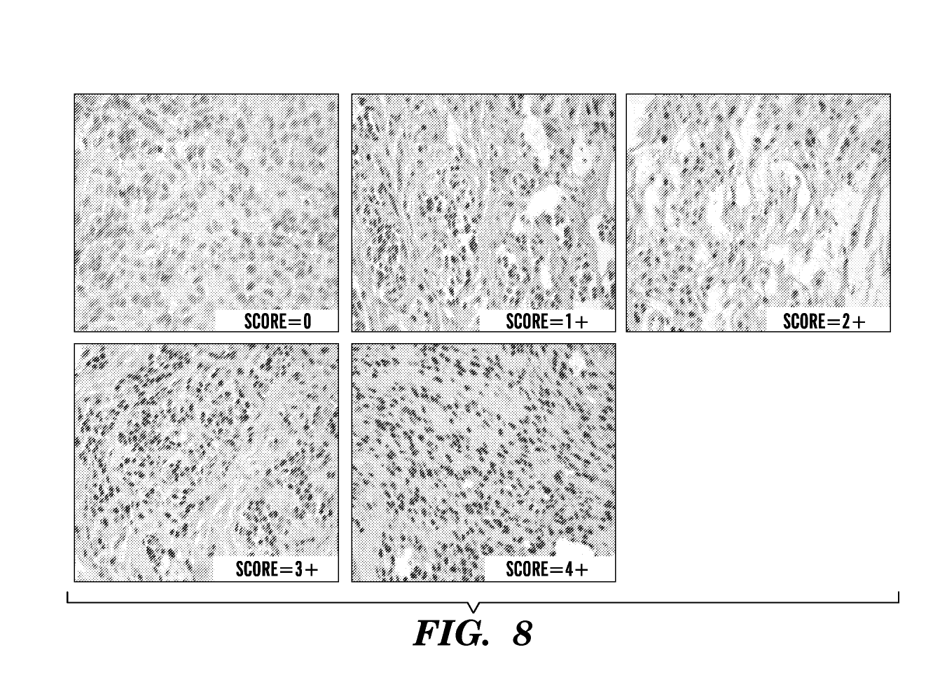

5-hmC levels were next examined by IHC using a melanoma progression tissue microarray (TMA) representing four major diagnostic tumor types: benign melanocytic nevus, primary cutaneous melanoma, melanoma metastases to lymph nodes and metastases to viscera (Kabbarah et al., 2010; Schatton et al., 2008). Consistent with the individual cases examined above (FIG. 1), the TMA confirmed significant 5-hmC loss in primary melanomas and metastatic melanomas compared with nevi (P<0.001; FIGS. 2A and 8, Tables 2 and 3). In two additional commercially available melanoma TMAs, there was significant loss of 5-hmC in melanomas compared to benign nevi (P=1.1×10⁻⁷) and loss in nodal compared to visceral metastases (P=0.016) (FIG. 2B). Taken together, these data further support “loss of 5-hmC” as a distinctive epigenetic event in melanoma, and suggest 5-hmC may represent a new epigenetic mark for melanoma recognition and progression.

The 5-hmC level was then correlated with critical melanoma staging parameters (tumor depth and mitotic rate) using the melanoma specimens from a clinically-annotated cohort including 70 superficial spreading and nodular melanomas (Table 4). There was a negative correlation between 5-hmC staining score and primary melanoma Breslow depth, a standard predictor of prognosis (FIG. 2C, r=−0.4, P=0.0005), as well as between 5-hmC level and mitotic rate (FIG. 2D, r=−0.23, P=0.054). Furthermore, 5-hmC levels were significantly reduced in melanomas with Breslow values of >1 mm compared to those with Breslow values of ≦1 mm (P<0.01), and melanomas with the presence of >1 mitosis (a current predictor of nodal metastasis) had less staining than those with ≦1 mitosis (P<0.05) (FIG. 2E and Tables 5 and 6). Similarly, 5-hmC levels in pathological stage 1 melanomas were significantly higher than in stage 2-3 melanomas (P<0.05), and were significantly lower in melanoma patients with ulceration (an important staging parameter) than those without ulceration (P<0.05) (FIG. 2E and Tables 5 and 6). The association between 5-hmC levels and the survival probability was further analyzed based on data for all 70 patients (Table 4). Importantly, Kaplan-Meier curves revealed that patients with 5-hmC-positive melanomas (staining score≧1) had significantly higher survival probabilities than patients with 5-hmC-negative melanomas (staining score=0) at diagnosis (FIG. 2F). Thus, loss of 5-hmC in melanoma has both diagnostic and prognostic value in these biospecimen cohorts.

### Genome-Wide Mapping of 5-hmC in Nevi and Melanomas Reveals a Demolished 5-hmC Landscape in the Epigenome of Melanomas

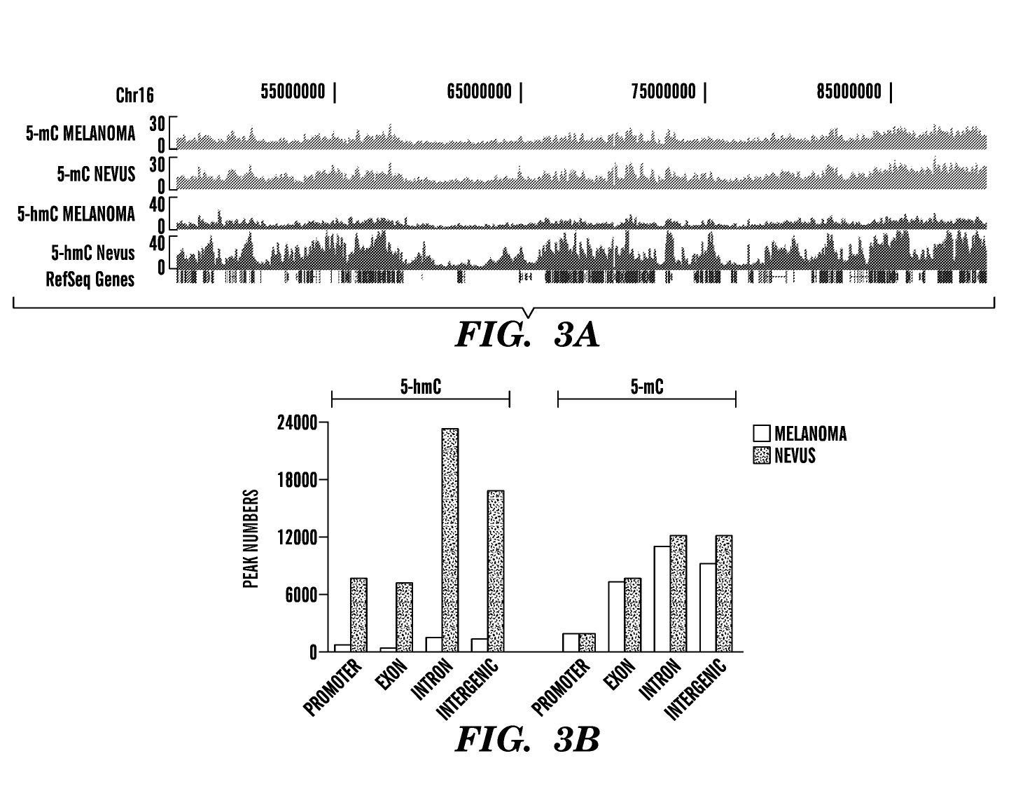

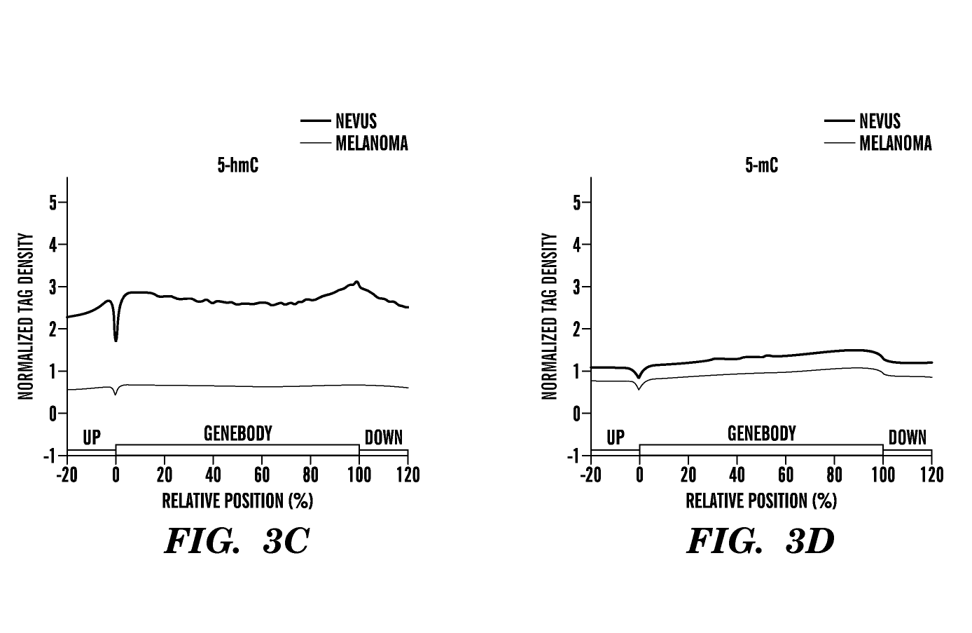

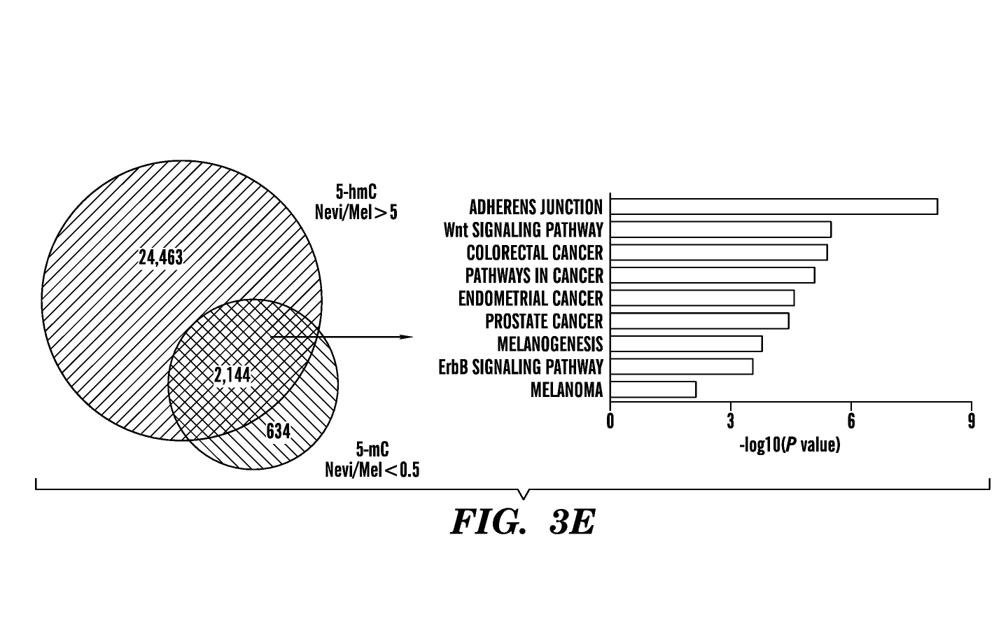

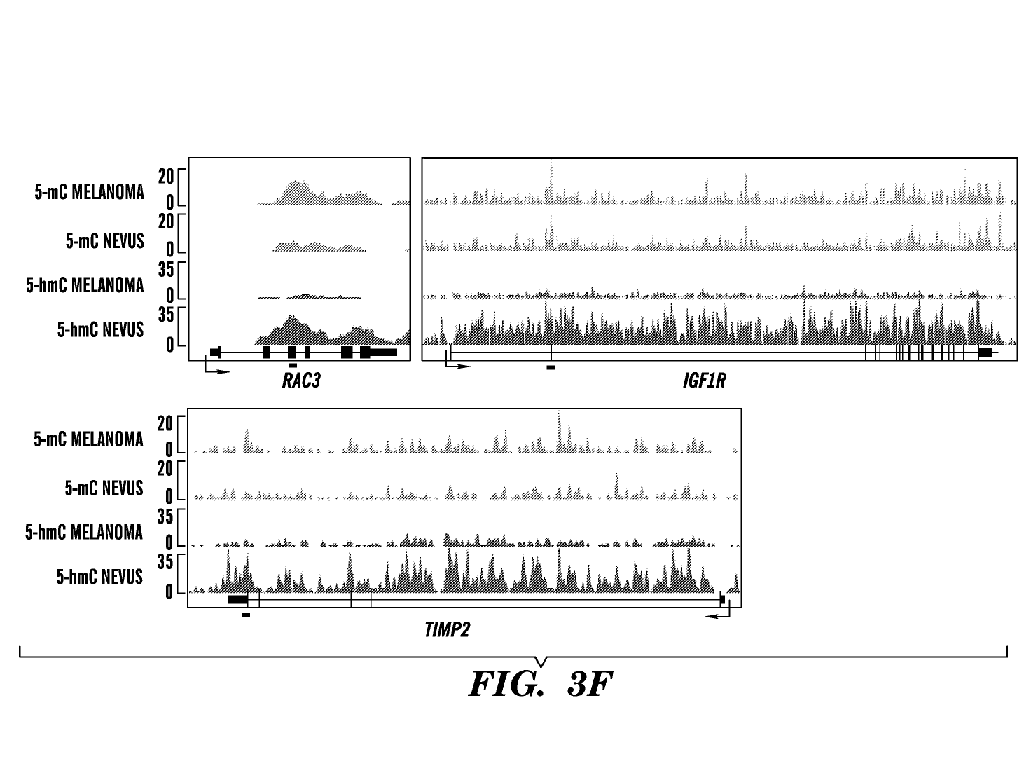

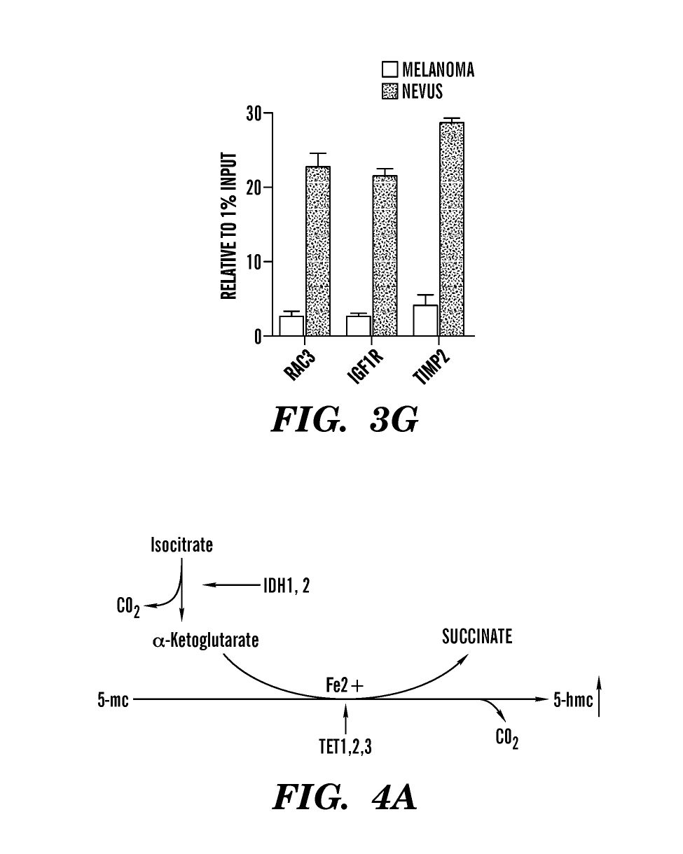

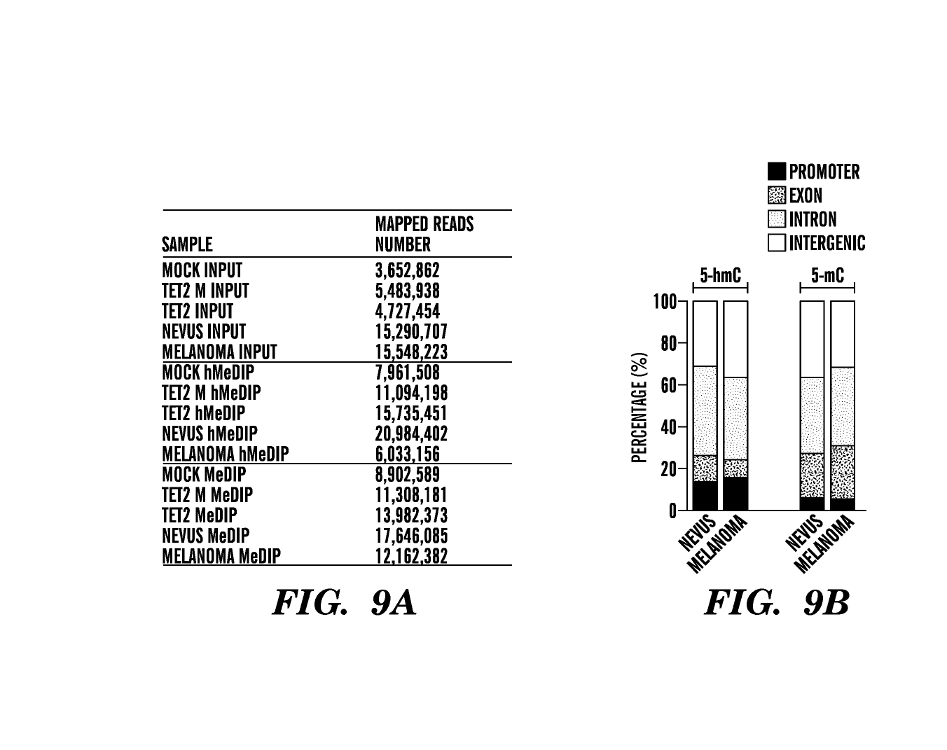

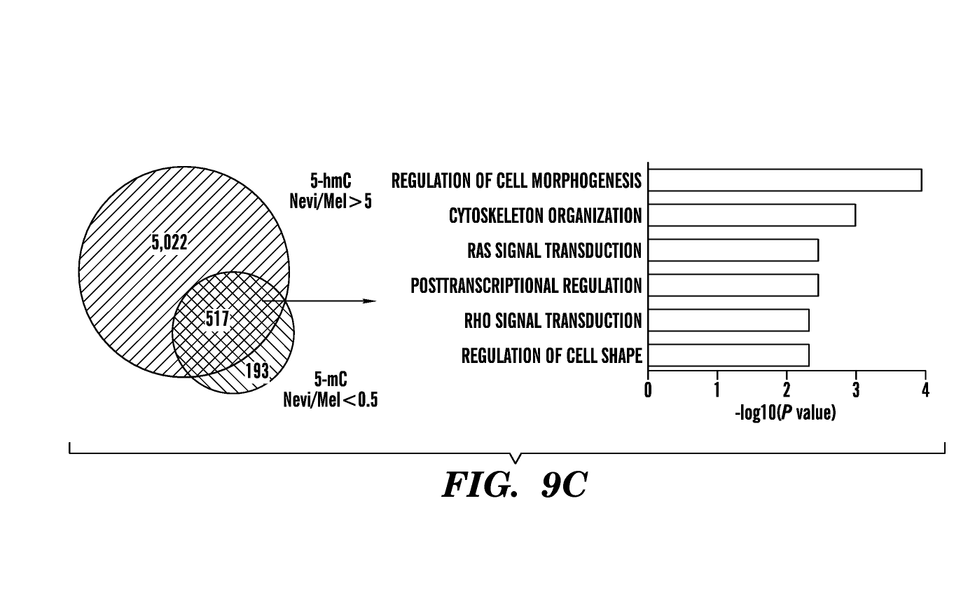

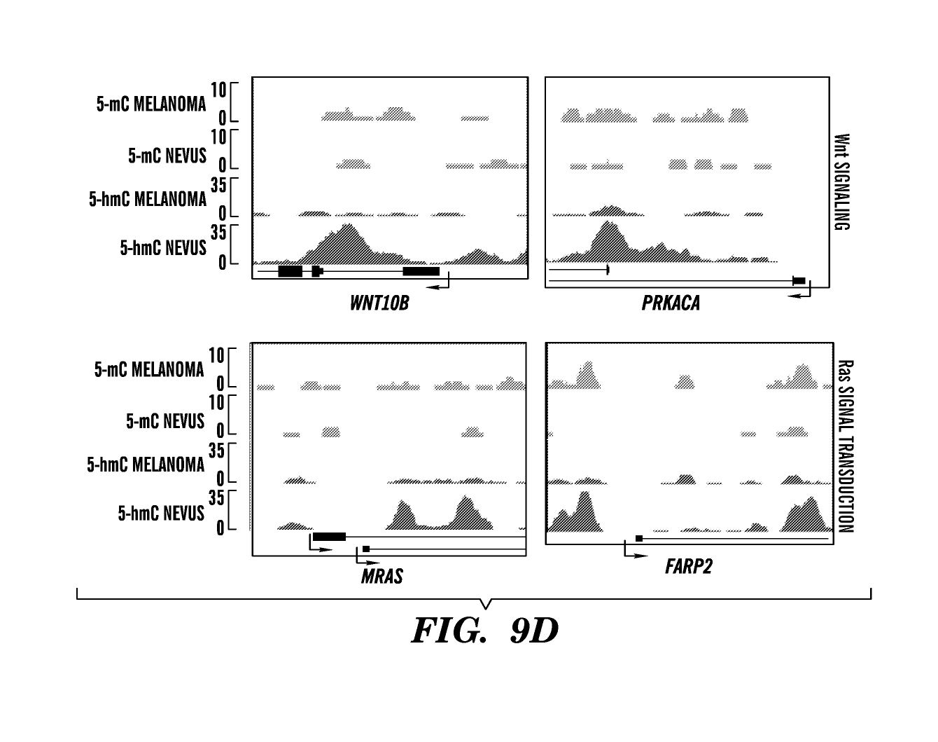

Whether 5-hmC loss in melanoma is genome-wide or loci-specific was then investigated. The 5-hmC level changes at specific genomic loci was first investigated by mapping the genome-wide 5-hmC distribution in nevus and melanoma tissues using a barcoded hydroxymethylated DNA immunoprecipitation (hMeDIP) approach coupled with deep sequencing (hMeDIP-seq) (Xu et al., 2011b) that permits quantitative comparisons of genome-wide changes of 5-hmC between nevi and melanomas (FIG. 9A). Similar to previous finding in mouse ES cells (Xu et al., 2011b), it was found that 5-hmC is associated with gene-rich regions in the nevus genome (FIG. 3A). Using MACS software (Zhang et al., 2008), a total 54,454 5-hmC peaks were identified in nevi (P<10⁻⁵, FDR<0.01, fold enrichment>10) among which more than half are located either in exons (13%) or introns (42.6%) and 13.9% are located at promoters (FIGS. 3B and 9B). These 5-hmC peaks are associated with 17,468 Refseq genes, in which 15,750 and 10,065 genes are modified by 5-hmC in gene bodies and promoters (−2k to +2k of transcriptional start sites (TSSs)), respectively. There are 8,347 Refseq genes that are modified by 5-hmC both at promoters and in gene bodies. However, only 3,362 5-hmC peaks were identified in melanomas using the MACS software with the same cut-off values as for nevi (FIGS. 3B and 9B). These peaks are only associated with 3,219 Refseq genes. Importantly, a significantly decreased 5-hmC level was observed within the averaged gene bodies and 20% of their up- and down-stream regions in melanomas in comparison to nevi (FIG. 3C). Indeed, 41,886 out of 54,454 (77%) total 5-hmC peaks were identified in nevi whose normalized 5-hmC densities were dramatically higher (>5-fold) than in melanomas. These 41,886 peaks are located in 15,240 Refseq genes at either promoters or gene bodies. Taken together, these analyses indicate that loss of 5-hmC is a genome-wide event during melanoma progression.

Whether the 5-mC genome-wide distribution is also altered in melanomas was then investigated. 33,374 and 28,830 5-mC peaks were identified in nevi and melanomas (P<10⁻⁵, FDR<0.01 and fold enrichment>5), respectively (FIGS. 3B and 9B). In contrast to 5-hmC, the genome-wide enrichment and distribution pattern of 5-mC is similar between nevi and melanomas, although a slight decrease of 5-mC in melanomas was observed (FIGS. 3B and 3D), which is consistent with the previously reported global DNA hypomethylation in melanomas (Tellez et al., 2009).

To determine whether gene specific changes of 5-hmC and 5-mC are associated with melanoma progression, 5-hmC and 5-mC tag densities were normalized in nevus and melanoma samples according to each input sequencing read, which permitted quantitative comparison of 5-hmC and 5-mC signal changes at specific genome loci. Since 5-hmC is converted from 5-mC by TET enzymes, the reasoning was that the decreased 5-hmC generation in melanomas would result in the accumulation of its substrate, 5-mC, at certain genomic regions compared to nevi. Indeed, 2,144 peaks were identified at which 5-hmC is dramatically higher (>5-fold) and 5-mC is significantly lower (>2-fold) in 3,401 Refseq gene bodies in nevi compared to melanomas (FIG. 3E). Importantly, KEGG pathway enrichment analysis for the 3,401 genes revealed that these genes are closely associated with various melanoma related pathways, such as adherens junction (P=6.05×10⁻⁹), Wnt signaling (P=1.67×10⁻⁷), pathways in cancer (P=8.65×10⁻⁷), and melanogenesis pathways (P=4.84×10⁻⁴) (FIG. 3E). As exemplified in FIG. 3F, RAC3, IGF1R and TIMP2 genes show decreased 5-hmC and increased 5-mC in gene bodies in melanomas compared with nevi, and the 5-hmC changes are further verified by conventional hMeDIP-qPCR assays (FIG. 3G). Similarly, 517 peaks were also identified at which 5-hmC is dramatically higher (>5-fold) and 5-mC is significantly lower (>2-fold) in 926 gene promoters in nevi compared to melanomas (FIG. 9C). Gene Ontology (GO) term and KEGG pathway analyses for the 926 genes also shows that they are involved in the regulation of cell morphogenesis (P=1.18×10⁻⁴), cytoskeleton organization (P=0.001), Ras protein signal transduction (P=0.00358), posttranscriptional regulation of gene expression (P=0.0036) (FIG. 9C) and Wnt signaling pathway (P=0.009). The 5-mC and 5-hmC densities at representative gene promoters are shown in FIG. 9D.

Thus, this study for the first time establishes the genome-wide map of methylome and hydroxylmethylome in nevus and melanoma and reveals the progressive loss of 5-hmC landscape in the epigenome from begin nevus to malignant melanoma. Thus, the demolished 5-hmC landscape is an epigenetic hallmark for melanoma.

### Down-Regulation of IDH2 and TET Family Members in Melanomas

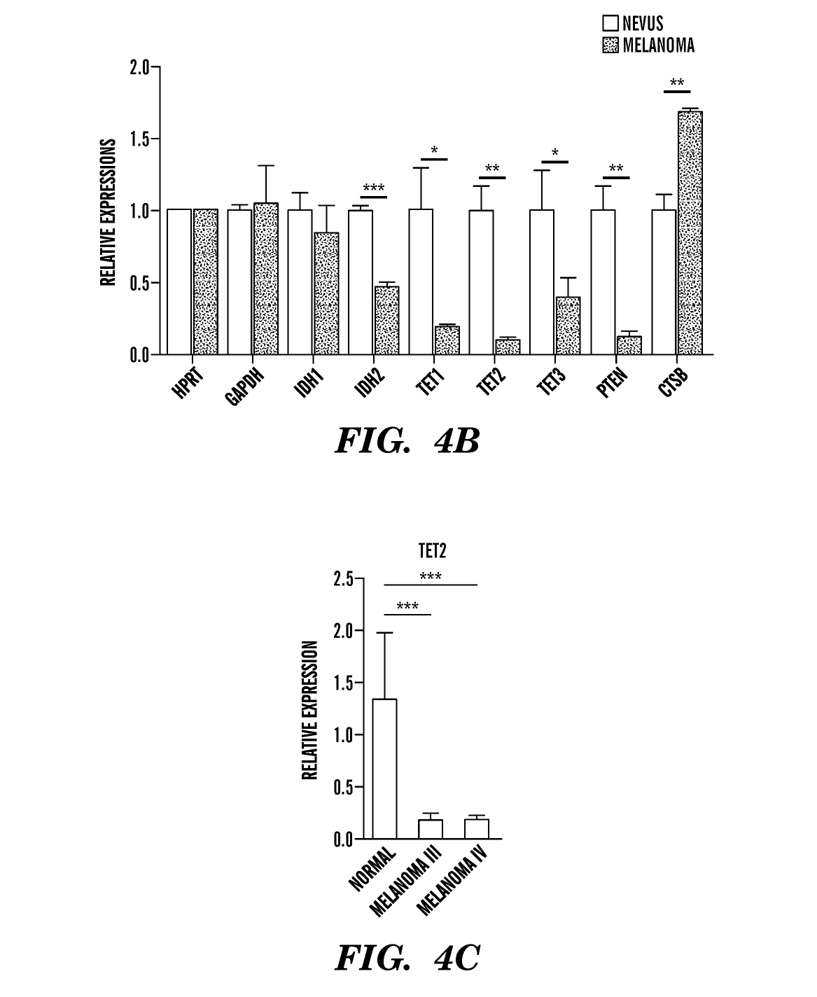

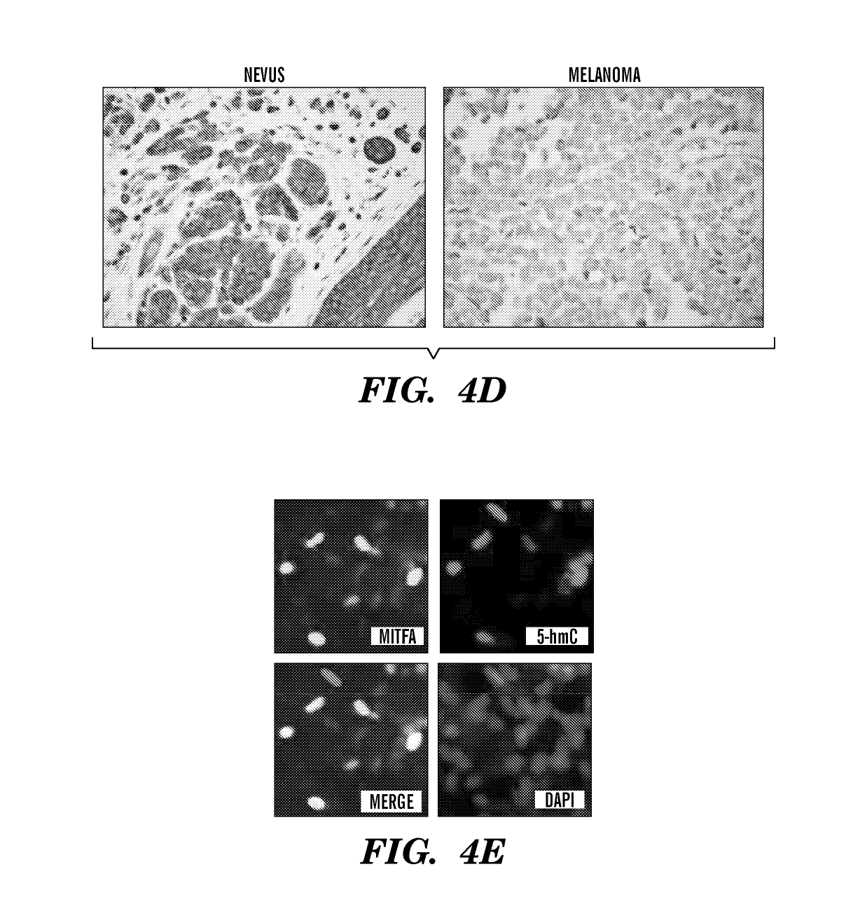

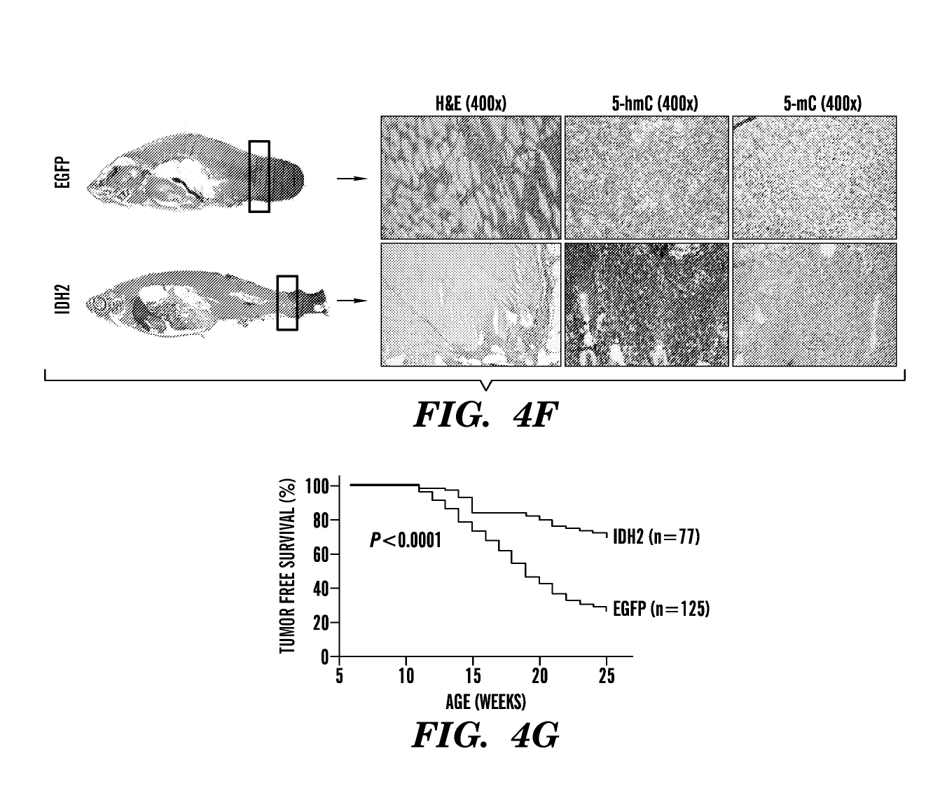

An investigation to determine the imminent cellular factors that are directly responsible for loss of 5-hmC in melanomas was then undertaken. While the TET family of 5-mC DNA hydroxylases are directly responsible for the generation of 5-hmC (FIG. 4A), the catalytic reaction requires cofactor α-KG (Ito et al., 2010; Tahiliani et al., 2009) which is mainly controlled by IDHs (Xu et al., 2011a). Therefore, it was hypothesized that IDH and/or TET family enzymes play a critical role in the establishment and maintenance of the 5-hmC landscape in the epigenome of melanocytes and their neoplasms, and down-regulation of these key enzymes may be responsible for the loss of 5-hmC in melanomas. To test this hypothesis, the expression levels of TET and IDH family genes were examined in both nevi and melanomas by RT-qPCR. While IDH1 has a similar expression level between nevi and melanomas, that IDH2 was found to be significantly down-regulated in melanomas (FIG. 4B). Strikingly, expression of all three TET genes was significantly lower in melanomas than in nevi, with the most dramatic decrease in the TET2 (FIG. 4B). As positive controls, decreased expression of tumor suppressor gene PTEN and increased expression of cathepsin B (CTSB) gene was observed in melanomas compared to nevi, while the expression of house keeping gene GAPDH was unchanged (Haqq et al., 2005; Riker et al., 2008; Talantov et al., 2005). Thus, the dramatically decreased expression of IDH2, TET1, TET2 and TET3 is specific and may reflect the down-regulated nature of these genes in melanomas. Furthermore, the decreased expression of TET2 in melanomas was confirmed via melanoma cDNA arrays (FIG. 4C), and the significantly decreased expression of IDH2 at the mRNA level was corroborated at the protein level by IHC staining (FIG. 4D). These data suggest that the diminished expression of IDH2 and/or TET family genes in melanomas may represent one of the molecular mechanisms underlying global loss of 5-hmC.

### Over-Expression of IDH2 in a Zebrafish Melanoma Model Increases 5-hmC Levels and Prolongs Tumor Free Survival

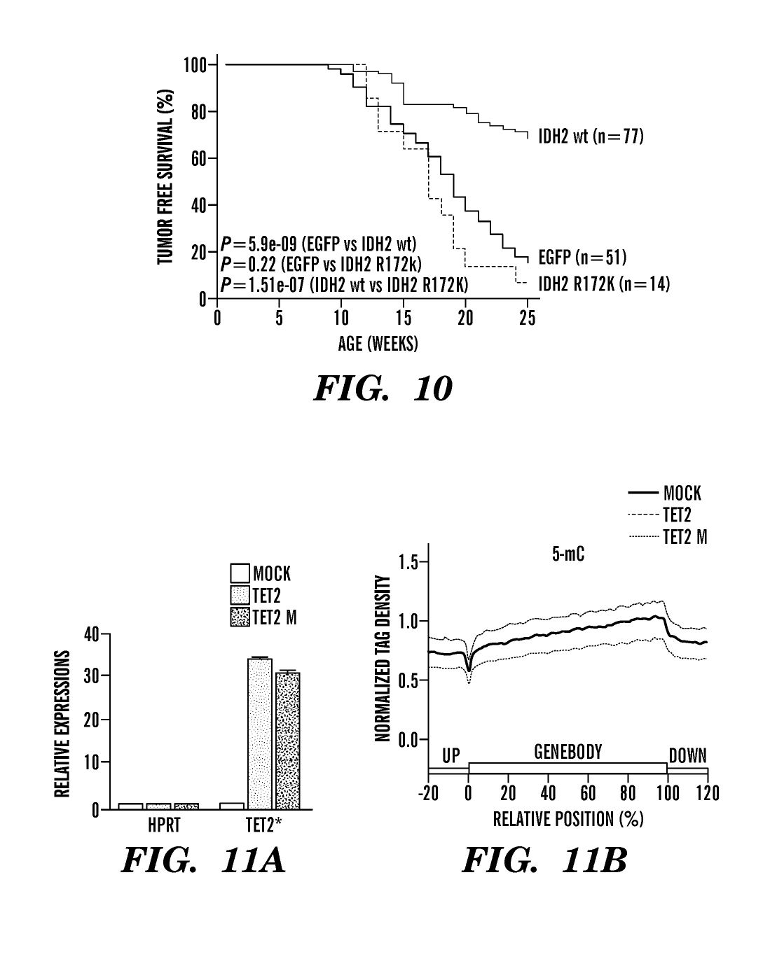

To test the hypothesis that over-expression of IDH2 may result in increased 5-hmC levels in melanoma and suppress tumor growth, a recently developed transgenic (Tg) zebrafish model for melanoma was employed, in which BRAF^{V600E} is expressed under the control of the mitfa gene melanocyte-specific promoter on a p53 mutant background (p53^{-/-}) (Ceol et al., 2011). While the 5-hmC level was high in normal zebrafish melanocytes shown by co-staining with melanocyte-specific mitfa (FIG. 4E), 5-hmC was barely detectable in melanomas of EGFP control animals (FIG. 4F upper panel). IDH2 over-expression greatly increases the 5-hmC level in melanomas compared to those in EGFP control animals (FIG. 4F lower panel), while over-expression of IDH2 R172K mutant does not shown any significant effects on the 5-hmC level. Strikingly, tumor incidence curve analysis revealed that IDH2 wt over-expressing group, but not IDH2 R172K over-expressing group, showed significantly increased tumor-free survival compared to the EGFP control group (P=7.8×10⁻⁹ by logrank test) (FIGS. 4G and 10). Collectively, these data suggest that the isocitrate dehydrogenase activity of IDH2 plays an important role in maintaining proper levels of 5-hmC in melanocytes and may function as a putative tumor suppressor for melanoma progression.

### Reestablishing the 5-hmC Landscape in the Epigenome of Human Melanoma Cells by Reintroducing TET2

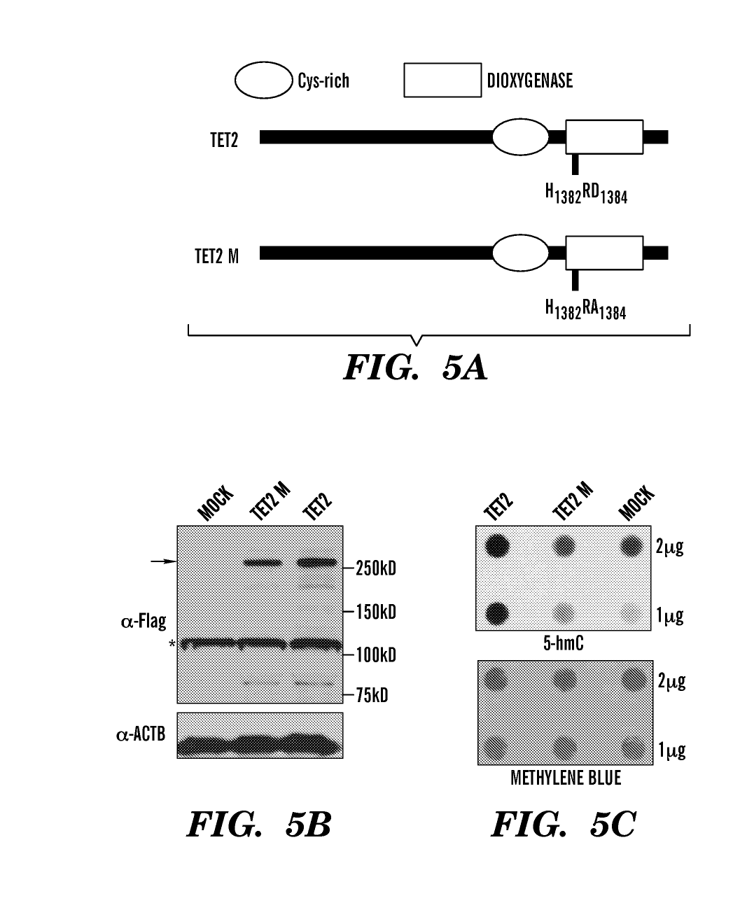

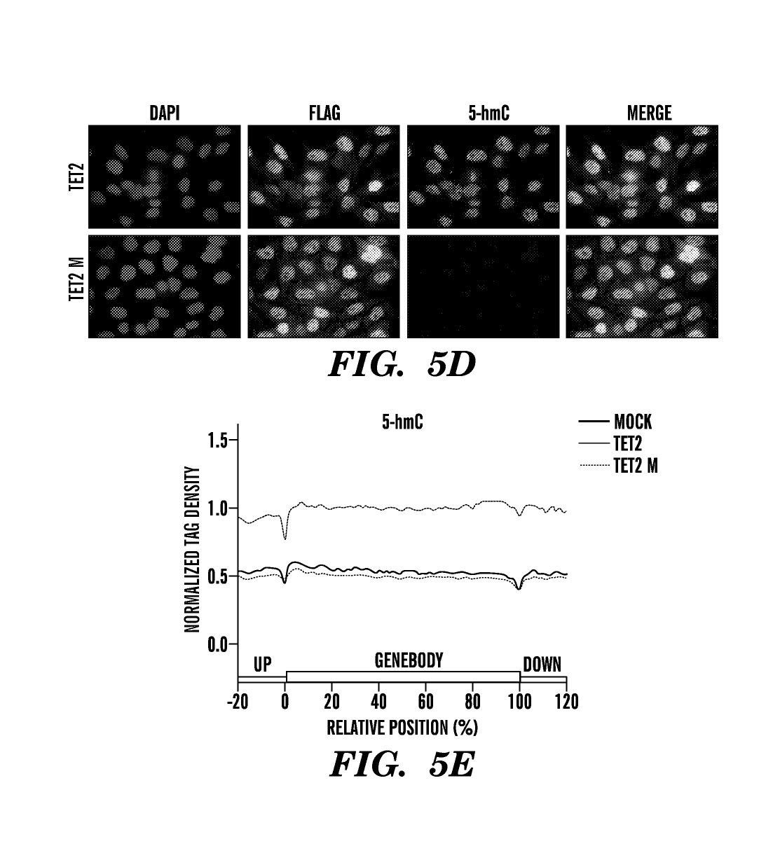

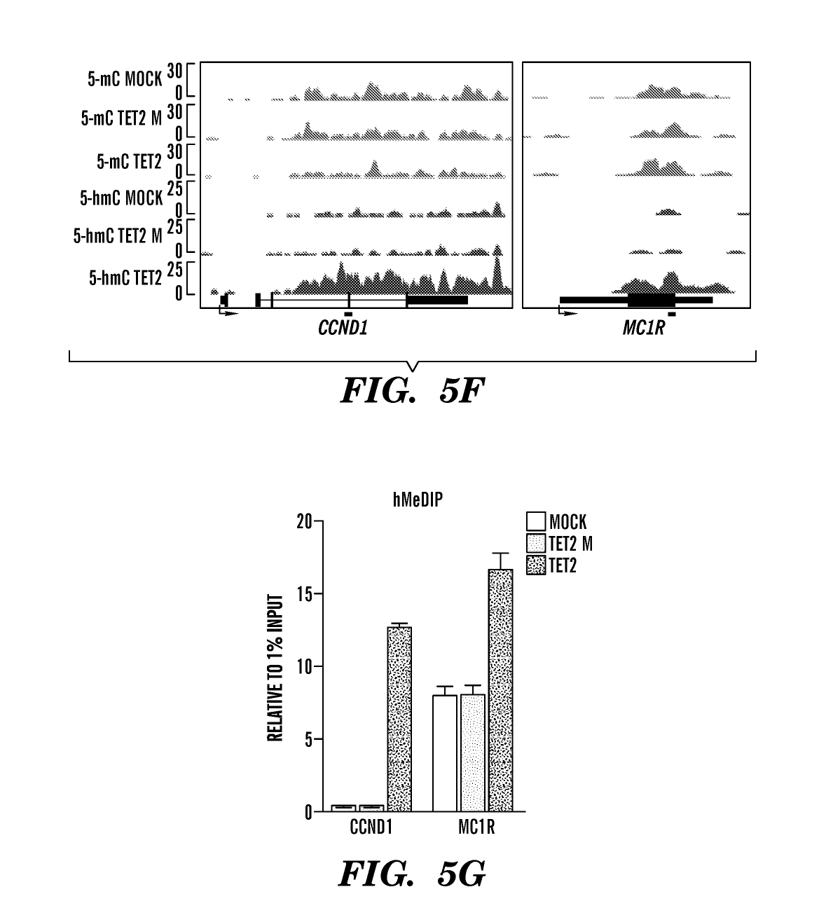

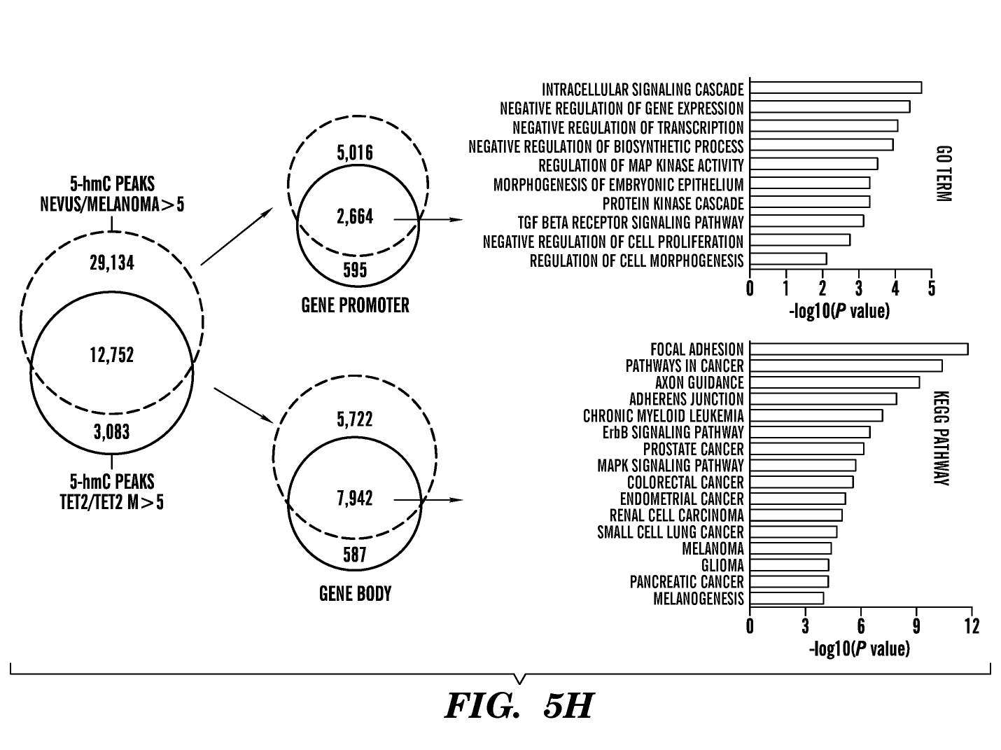

Whether reintroducing TET2, the most dramatically decreased TET family gene in human melanomas can rescue the demolished 5-hmC landscape in melanoma cells was then investigated. To exclude the 5-mC hydroxylase-independent function of TET2, pure monoclonal A2058 stable cell lines over-expressing flag-tagged full length wt TET2 or the iron-binding site (H₁₃₈₂RD₁₃₈₄) disrupted catalytically-inactive mutant (TET2 M) were generated (FIG. 5A), as well as the vector only control (Mock). The over-expression of full-length TET2 and TET2 M was verified by Western blot and RT-qPCR assays (FIGS. 5B and 11A). The global increase of 5-hmC levels in TET2 over-expressing cells, but not TET2 M over-expressing cells, compared to Mock cells by dot-blot and IF assays was confirmed (FIGS. 5C and 5D).

The genome-wide maps of 5-mC and 5-hmC in Mock, TET2- and TET2 M-over-expressing melanoma cells by MeDIP-seq and hMeDIP-seq as described above (FIG. 9A). While only marginal changes in 5-mC levels among Mock, TET2- and TET2 M-over-expressing melanoma cells were observed (FIGS. 9A and 11B), TET2 over-expressing melanoma cells showed re-establishment of the 5-hmC landscape in their epigenome (FIG. 5E). No significant 5-hmC level differences were found between Mock and TET2 M cells. As exemplified by CCND1 and MC1R genes (FIG. 5F), deep sequencing data were confirmed by conventional hMeDIP-qPCR assays (FIG. 5G).

Further bioinformatic analyses identified 15,835 peaks in TET2 over-expressing cells whose 5-hmC densities are dramatically higher (>5-fold) than in TET2 M cells. 80% (12,752/15,835) of these peaks overlap with a significant portion (30%, 12,752/41,886) of 5-hmC peaks whose 5-hmC levels are dramatically lower (>5-fold) in melanomas than nevi (FIG. 5H, left panel), suggesting that the 5-hmC landscape in those genomic regions can be reestablished by reintroducing TET2 in human melanoma cells. These overlapping 5-hmC peaks were further analyzed according to their locations (promoter or gene body) at associated genes. 2,664 Refseq genes were identified whose promoters have consistently higher 5-hmC densities in nevi and TET2 over-expressing cells compared to melanomas and TET2 M cells, respectively (FIG. 5H, upper middle panel). GO term analysis reveals that these 2,664 genes are mainly associated with intracellular signaling cascade, regulation of gene transcription, cell proliferation and morphogenesis (FIG. 5H, upper right panel). Similarly, 7,942 Refseq genes whose gene bodies have consistently higher 5-hmC densities in nevi and TET2 over-expressing cells were identified compared to melanomas and TET2 M cells, respectively (FIG. 5H, lower middle panel). Importantly, KEGG pathway analysis shows an impressive functional association of these genes with various cancer-related pathways, such as focal adhesion (P=1.9×10⁻¹²), pathways in cancer (P=4.32×10⁻¹¹), adherens junction (P=7.94×10⁻¹⁰), melanoma (P=5.39×10⁻⁵) as well as ErbB (P=3.86×10⁻⁷) and MAPK (P=2.29×10⁻⁶) signaling pathways (FIG. 5H, lower right panel).

In sum, these biochemical and genome-wide analyses suggest that reintroducing wt TET2, but not the TET2 catalytically inactive mutant, is able to significantly increase the global 5-hmC level and reestablish, at least in part, the 5-hmC landscape in the epigenome of human melanoma cells, which may subsequently affect several biological processes, such as cancer progression.

### Tumor Invasion and Growth is Suppressed by TET2-Mediated Reestablishment of the 5-hmC Landscape in Melanoma Cells

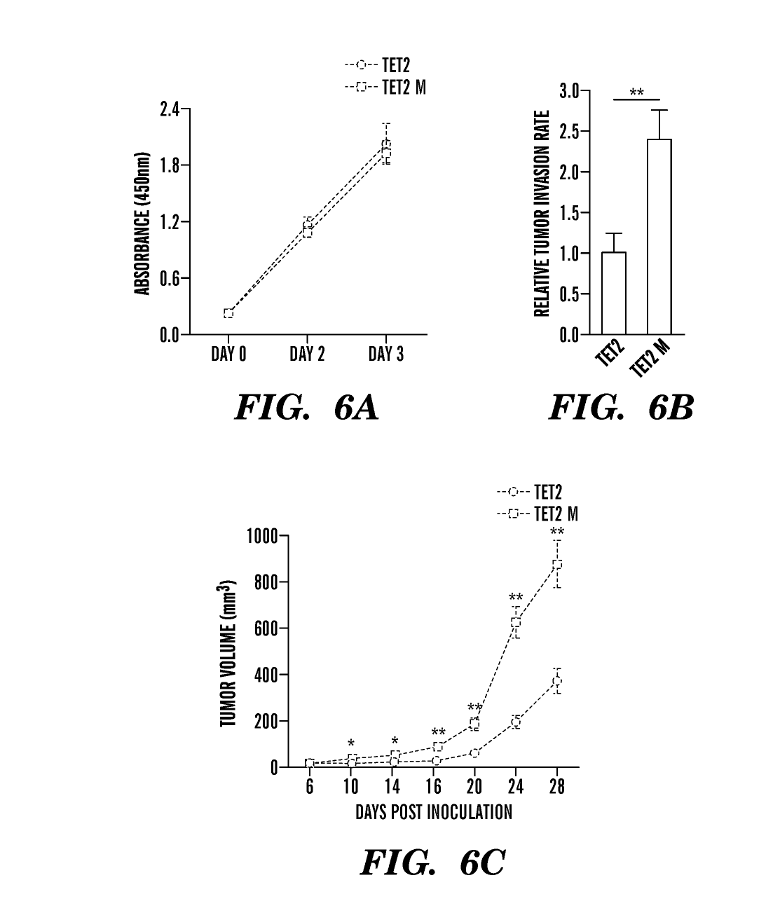

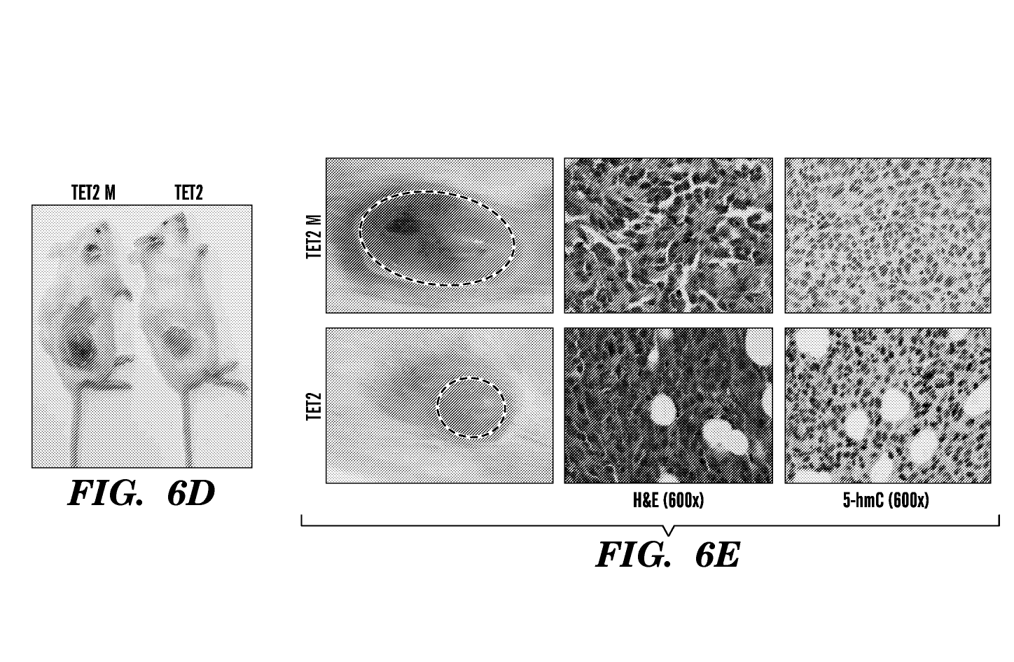

To determine the biological consequence of TET2-mediated reestablishment of the 5-hmC landscape in melanoma cells, the invasive ability of A2058 cells over-expressing TET2 and TET2 M was first compared by in vitro Matrigel assay. While these two cell lines have similar in vitro proliferation rates (FIG. 6A), TET2 over-expressing cells show a significantly lower invasion rate than TET2 M cells (FIG. 6B). Next, the putative tumor suppressor role of 5-hmC and TET2 was investigated using in vivo xenograft assays. TET2- and TET2 M-over-expressing melanoma cells were injected into NSG mice and tumor growth was monitored. TET2 over-expressing melanoma cells gave rise to significantly smaller tumors compared to TET2 M melanoma cells (FIGS. 6C and 6D). NSG mice injected with Mock melanoma cells showed no significant differences in tumor growth compared to TET2 M over-expressing melanoma cells. Importantly, 5-hmC IHC staining of the xenografted tumor sections showed that smaller tumors derived from TET2 over-expressing cells retained relatively high levels of 5-hmC (FIG. 6E, right panels). Thus, these data highlight a potential tumor suppressor role of increased 5-hmC levels meditated by the enzymatic activity of TET2 in melanoma.

### Discussion

Here it is reported that “loss of 5-hmC” is a distinctive epigenetic event of neoplastic progression in melanoma that correlates with clinical relapse-free survival and melanoma staging parameters. Thus, 5-hmC holds promise as a putative molecular biomarker with predictive and prognostic value. The present study also for the first time illustrates the genome-wide 5-hmC landscape of benign nevi and melanomas and reveals the strikingly demolished 5-hmC levels and distribution along the epigenome of melanomas in comparison with benign nevi. Furthermore, loss of 5-hmC in melanoma is caused, at least in part, by the decreased expression of key enzymes, IDH2 and TET family proteins, controlling 5-hmC production. In relevant animal models, increase in 5-hmC levels via either IDH2 or TET2 overexpression is shown to suppress tumor invasion and growth and improve tumor free survival. Taken together, the present study provides multiple layers of evidence to support that genome-wide “loss of 5-hmC” is a new epigenetic hallmark of melanoma with diagnostic and prognostic advantages over global DNA hypomethylation, a recognized epigenetic mark of cancer. Of clinical and therapeutic significance, the present study also opens a new avenue for cancer prevention by targeting the cellular and biochemical pathways that can re-establish 5-hmC levels and landscape in melanoma.

The IHC staining approach to detect 5-hmC may have practical applications clinically. Similar loss of 5-hmC in other solid tumors such as breast, ovarian, and colon carcinoma (data not shown) has been observed using the same methods (Haffner et al., 2011; Yang et al., 2012). The anti-5-hmC antibody-based IHC strategy could lead to the development of a new, simple, sensitive and practical adjuvant diagnostic assay. Future studies focusing on borderline lesions and with sufficient clinical outcome annotation are now indicated to evaluate the utility of “loss of 5-hmC” as a novel diagnostic and prognostic tool.

The high level of 5-hmC in differentiated and benign nevomelanocytes raises an intriguing question as to the nature of the biological role of 5-hmC in melanocyte differentiation, self-renewal and malignant transformation. Studies of embryonic stem (ES) cells indicate that 5-hmC may be involved in the regulation of cell differentiation and lineage commitment (Ito et al., 2010; Xu et al., 2011b). Until now, skin tissue-specific 5-hmC distribution and genome-wide mapping of 5-hmC in cancer have not been well studied. These findings of a high level of 5-hmC in mature melanocytes and benign nevi, as well as a significantly lower level of 5-hmC associated with melanoma, provide new insights supporting a role of 5-hmC in pathways fundamental to cellular differentiation and de-differentiation. It is postulated that the well-controlled dynamic level of 5-hmC during transition from ES cell to melanocyte progenitor to terminally-differentiated melanocyte is a novel epigenetic signature of melanocyte differentiation, the perturbation of which may lead to the induction of oncogenic pathways underlying melanoma progression.

There are several ways to influence 5-hmC levels in cells (FIG. 4A). Presumably, dysfunction of TET and/or IDH enzymes, two key factors involved in 5-hmC generation, would greatly reduce 5-hmC generation. 10% of melanomas (4/39) harbor an IDH1 or IDH2 mutation Shibata et al., 2011), whereas no TET mutations have been reported in melanoma. The low penetration of IDH and TET mutations in melanoma constitutes robust evidence that other cancer pathways inactivating these 5-hmC-generating enzymes must play a major role in down-regulation of 5-hmC. Herein, the significant decrease in TET1, TET2, TET3 and IDH2 gene expression is demonstrated in melanomas compared to benign nevi, which suggests that insufficient enzymes required for the conversion of 5-mC to 5-hmC may account for one of the molecular mechanisms underlying global “loss of 5-hmC” in melanomas.

In support of this hypothesis, it is demonstrated that increasing 5-hmC levels and partially re-establishing the 5-hmC landscape in melanoma cells by restoring expression of active TET2 enzyme but not the catalytically inactive TET2 mutant, significantly suppresses tumor growth in a murine human melanoma xenograft model. Importantly, this study excludes the possibility that the tumor-suppressive effect is due to the over-expression of TET2 itself. Rather, such an effect is due to elevated levels of 5-hmC on the genes important for key cellular processes. Moreover, a forced increase in 5-hmC is demonstrated in an established zebrafish melanoma model via over-expression of IDH2 wt, but not IDH2 R172K mutant, to significantly suppress tumor growth and prolong tumor-free survival. These data suggest that IDH2, but not IDH1, is specifically down-regulated in melanomas and that the wt IDH2 acts as a putative tumor suppressor in the zebrafish melanoma model although the IDH family of enzymes have been considered candidate oncogenes in various tumors (Ward et al., 2010; Wrzeszczynski et al., 2011). Nonetheless, while the nature of the putative tumor suppressor function of IDH2 in melanoma and other tumor types warrants future investigations, a dramatic increase in the global 5-hmC level is the most pronounced epigenetic alteration observed both in the TET2 and IDH2 over-expressing melanoma animal models. Thus, these data demonstrate that high levels of 5-hmC and the appropriate 5-hmC landscape in the epigenome of melanocytes and nevus cells, both potential melanoma progenitors, play a role in preserving the integrity of these indolent cells and in preventing melanoma initiation and progression. This study supports the novel concept that an elevated level of 5-hmC can serve as a distinctive epigenetic molecular beacon for the reversal of an aggressive melanoma phenotype. It further indicates that particular TET and IDH family enzymes have putative tumor suppressor functions in melanoma progression, and spontaneously targeted down-regulation or inactivation of multiple key enzymes in 5-hmC generating pathway is one of the epigenetic mechanisms underlying melanoma development.

The present study also attempts to address the molecular mechanisms directly linking the gene specific 5-hmC loss to melanoma formation. Genome-wide mapping and comparative analyses of 5-mC and 5-hmC landscape in benign nevi, primary melanomas, MOCK, TET2- and TET2 M-over-expressing melanoma cells indicated that a program of genes involving various cancer pathways display significant reduction of 5-hmC in comparisons between benign nevi and melanoma, which can be reversed by over-expression of active TET2 but not inactive TET2 M. However, simple correlations between significant loss of 5-hmC and expression of associated genes was not found because a reduced 5-hmC level is associated with both up- and down-regulated genes in melanoma compared with nevi. This is not surprising since the complex roles of 5-hmC in gene transcription regulation in mouse ES cells has been shown (Ficz et al., 2011; Xu et al., 2011b). Thus, understanding the intricate relationship between the regulation of 5-hmC and associated gene transcription remains a challenge in 5-hmC biology (Cimmino et al., 2011; Wu and Zhang, 2011). Of note, a subset of genes showing significant 5-hmC level decreases and simultaneous 5-mC level increases was identified in melanomas compared to nevi. The strong association of this subset of genes with various cancer pathways suggests that gene-specific 5-hmC loss may partially contribute to the abnormal DNA methylation pattern in the epigenome of melanoma that has been linked to the progression of various cancers. However, there are a portion of genes showing significant reduction in 5-hmC levels but no obvious 5-mC level changes in melanomas compared to nevi, suggesting that other independent molecular mechanisms, such as 5-hmC-mediated regulation of cell division/replication, cell differentiation/senescence and/or genome/epigenome instability, may also be involved in linking the loss of 5-hmC to melanoma progression. Future studies aimed at identifying these potential 5-hmC-related mechanisms should enhance insights into 5-hmC function in melanoma formation and progression.

Finally, with melanoma as a paradigm of aggressive cancer, this study provides important insight for future functional studies of 5-hmC in cancer biology. Increasing 5-hmC levels via over-expressing TET2 reversed the genome-wide 5-hmC distribution from the global 5-hmC loss pattern in melanoma toward a benign nevus-like pattern. More importantly, the phenotype of melanoma was rescued by increasing 5-hmC levels via over-expressing either TET2 or IDH2 in animal models. Thus, “loss of 5-hmC” in melanoma progression is a fundamental epigenetic event that provides proof-of-principle that key factors in the 5-hmC generating pathway can be therapeutically targeted to restore 5-hmC in human melanoma, thus revealing new strategies for the design of melanoma treatment.

### Materials and Methods

### Immunohistochemical (IHC) Staining

Immunohistochemical studies employed 5-μm sections of formalin-fixed, paraffin-embedded tissue. Slides were de-paraffinized and rehydrated, and after antigen retrieval, were placed in 2N HCl for 30 minutes, rinsed in distilled water and placed in 100 mM Tris-HCl pH8.5 for 10 minutes. All were stained on the Dako Autostainer (Dako Corporation, Carpinteria, Calif.) using the EnVision (Dako) staining reagents. Sections were incubated for 60 minutes with either rabbit-anti-5-hmC at 1:10,000 (Active motif) or mouse-anti-5-mC at 1:500 (Eurogentec) and then incubated with the EnVision+Dual Link (Dako) detection reagent for 30 minutes. Sections were washed, treated with a solution of diaminobenzidine and hydrogen peroxide (Dako) for 10 minutes, and after rinsing, a toning solution (DAB Enhancer, Dako) was used for 2 minutes to enrich the final color.

### Glucosylation of Genomic 5-hmC

Genomic DNA (700 ng) purified from benign nevi or melanomas was incubated with 1 μl of T4 Phage β-glucosyltransferase (NEB) and 1 μl of UDP-glucose [6-3H] (ARC) in 1×NEB buffer 4 at 37° C. overnight, followed by protease K digestion. DNA was purified and radioactivity was measured by Beckman Coulter scintillation counter LS6500.

MeDIP-seq and hMeDIP-seq

Genomic DNA of human melanomas, nevi and human melanoma cell lines was purified and sonicated. Illumina barcode adapters were ligated before hMeDIP. Adaptor-ligated DNA (5 μg) was denatured and incubated with 3 μl of 5-hmC antibody (Active Motif) or 5 μg of 5-mC antibody (Eurogentec) at 4° C. overnight. Antibody-DNA complexes were captured by protein A/G beads. The immunoprecipitated DNA was purified and sequenced followed by standard Illumina protocols (Xu et al., 2011b). Read sequences were mapped to the human genome (hg19) using ELAND v2 in the CASAVA (Illumina, v1.6) package. Significantly enriched regions were determined by Model-based Analysis of ChIP-Seq (MACS) package (Zhang et al., 2008). GO term and KEGG pathway analyses were performed by the database for annotation, visualization and integrated discovery (DAVID) programs (Huang et al., 2009).

### NSG Mice Melanoma Xenograft Assay

NOD/SCID interleukin-2 receptor (IL-2R) γ-chain null (NSG) mice were purchased from The Jackson Laboratory (Bar Harbor, Me.) and maintained under defined conditions in accordance with institutional guidelines. Experiments were performed according to approved experimental protocols. For tumorigenicity studies, MOCK, wt TET2 or TET2 M A2058 melanoma cells were injected subcutaneously into the flanks of NSG mice (1×10⁶/injection). Tumor growth was assayed as a time course (Schatton et al., 2008) for the duration of the experiment or until excessive tumour burden or disease state required protocol-stipulated euthanasia.

### IDH2 Over-Expression in a Zebrafish Melanoma Model

The miniCoopR assay was performed as previously described (Ceol et al., 2011). Transgenes were expressed in zebrafish melanocytes in the background of a stably-integrated BRAF^{V600E} transgene and a p53 loss-of-function mutation. The background also contained a mitfa loss-of-function mutation, which blocked melanocyte development. Transgenes were coupled, via the miniCoopR vector, to a rescuing mitfa gene, ensuring that rescued melanocytes also expressed the transgene being tested. IDH2 was cloned into the miniCoopR vector, and the resulting miniCoopR-IDH2 construct was injected into single-cell zebrafish embryos. Transgenic animals were selected and melanoma onset measured weekly as compared to miniCoopR-EGFP control animals. Animals with melanomas were isolated and tumors allowed to progress for two weeks prior to being sacrificed. Tumors were formalin fixed, embedded and sectioned transversely to assess invasion.

### Interpretation of IHC

Positive staining was defined as a dark brown staining pattern, confined to the nuclear region. Scant or fine granular background staining or no staining was considered as negative. Nuclear positivity for 5-hmC was evaluated only in areas of sub-epidermal invasive melanoma. The IHC staining was interpreted and scored on a scale according to Table 8. The status of 5-hmC staining was assessed by 2 researchers without knowledge of the clinical and pathologic features of the cases. Negative control array was concurrently run showing <1% nuclear staining in all specimens. All specimens were evaluated according to the 0-4 grading criteria based on percentage of 5-hmC positive cell counts (Table 2 left column and FIG. 8). Staining intensity was not factored into the analysis here because not all samples in the TMA contained keratinocytes as controls for comparison (Table 2 right column). Two commercially available melanoma TMAs were analyzed in this study. One TMA (CK2, Imgenex, Inc.) contained 52 examinable specimens, with 37 primary and 15 metastatic melanomas. The other TMA (ARY, US Biomax, inc.) contained 79 evaluable specimens including 27 primary melanomas and 27 melanoma metastases. For clinical cohort study, a two tier grading system with the staining score of both counts (averaged over 3 high power fields) and staining intensity (averaged over 3 high power fields), and the product of these two values were applied due to many of the cores without epidermis present (Table 2).

### Laser Capture Micro-Dissection and DNA Isolation

Sections of each sample (8 μm thick) were mounted on both positively charged glass microscope slides (Richard Allen Bond-Rite) for pathological examination and membrane slides (Molecular Machines Industries) for laser capture microdissection, respectively. Glass and membrane slides were hematoxylin- and eosin-stained utilizing standard protocols. Laser capture microdissection was performed on either a Molecular Machines Industries or Palm system. Briefly, target cell populations (either benign nevic cells or melanoma tumor cells) were dissected with the laser microbeam and then catapulted with a single laser shot into the lid of a microcentrifuge tube. The collected cells were then recovered in 20 μl of lysis buffer. After lysis at 37° C. for 16 hours, the sample was spun down by centrifugation, and was inactivated with proteinase K at 70° C. for 10 minutes.

### RT-qPCR

Total RNA of human melanomas and benign nevi were extracted by RNAeasy kit (Qiagene) and the cDNAs were synthesized by SuperScript III first strand kit (Invitrogen). The human melanoma cDNA arrays were obtained from Origen (Cat. No.: MERT301). Relative gene expression was normalized to HPRT. Primers used in qPCR are listed in Table 8.

### TET2 and TET2 M Stable Cell Lines

Human TET2 gene (NM_{—}001127208.2) was amplified from HEK293T cells and subcloned into pOZN vector. To make TET2 enzymatic activity dead mutant (TET2 M), TET2 iron binding site H₁₃₈₂RD₁₃₈₄ was mutated to Y₁₃₈₂RA₁₃₈₄ using QuickChange Site-Directed Mutagenesis Kit (Stratagene). MOCK, TET2 or TET2 M containing retrovirus was generated and the virus-infected A2058 cells were selected as previously described (Nakatani et al., (2003). Methods in Enzymology (Academic Press), pp. 430-444. 2003). To obtain pure monoclonal stable cell lines, the selected cells were further serially diluted and the expression of full length TET2 and TET2 M was verified by RT-qPCR at mRNA level and Western Blot at protein level.

### In Vitro Tumor Invasion Matrigel Assay

Cells (2.5×10) were seeded into the upper compartments of either BD BioCoat Growth Factor Reduced Matrigel Invasion Chambers or BD BioCoat Control Inserts (BD Biosciences, USA), and DMEM supplemented with 10% FBS was added to the lower compartment according to the manufacturer's instructions. The invasion chambers and control inserts were incubated for 8-24 h at 37 degree in a humidified atmosphere containing 5% CO₂. After incubation, the non-invading cells were removed from the upper surface of the membrane by gentle scrubbing, and the cells on the lower surface of the membrane were fixed in 10% formalin and stained with hematoxylin Grill#1 and 0.1% ammonium hydroxide. Cell counting was facilitated by photographing the membrane through the microscope at the area of highest cell density, and triplicate membranes were counted at 200 magnifications per experiment. The percent invasion was calculated by dividing the mean number of cells that invaded through the Matrigel insert membrane by the mean number of cells that migrated through the control insert.

### Cell Proliferation Assay

Cells (2×10³) were seeded into 96-well plates. Alive cell numbers were counted at Day 0, 2 and 3 using Cell Counting Kit-8 (Dojindo Molecular Technologies, Inc., Cat. No: CK04-05).

### Statistical Analysis

The statistical analysis was performed using SAS software.

### Accession Numbers

Sequencing data have been deposited to GEO (accession number GSE38231). The contents of GEO accession number GSE38231 are incorporated herein by reference in their entirety.

TABLE 1
List of epi-mark antibodies, Related to FIG. 1

| Antibody | Function |
|---|---|
| Anti-5-hydromethylcytosine | DNA-methylation |
| Anti-5-methylcytosine | DNA-methylation |
| Anti-H3K4 me1 | Histone modification |
| Anti-H3K4 me2 | Histone modification |
| Anti-H3K4 me3 | Histone modification |
| Anti-H3K9 me3 | Histone modification |
| Anti-H3K36 me3 | Histone modification |
| Anti-H3K36 me2 | Histone modification |

TABLE 2
The scoring systems for grading 5-hmC
IHC staining, Related to FIGS. 2 and 8

| Score system | Positive cell | Score system | Nuclear staining |
|---|---|---|---|
| of cell count | number | of Intensity | positivity |
| 0 | <1% | 0 | no staining |
| 1+ | 1-9% | 1+ | lighter than nuclei of |
| | | | keratinocytes |
| 2+ | 10-24% | 2+ | equivalent to nuclei of |
| | | | keratinocytes |
| 3+ | 25-74% | 3+ | darker than nuclei of |
| | | | keratinocytes |
| 4+ | >74% | | |

TABLE 3
5-hmC staining analyses of SPORE Melanoma
Progression TMA, Related to FIG. 2

| | Case number | Positive Cell Count* |
|---|---|---|
| Benign Nevus | 66 | |
| Thin Nevus | 21 | 2.33 ± 0.19 |
| Thick Nevus | 45 | 2.60 ± 0.11 |
| Primary Melanoma | 98 | |
| Thin primary melanoma | 60 | 0.58 ± 0.11 |
| Thick primary melanoma | 38 | 0.35 ± 0.06 |
| Metastatic Melanoma | 76 | |
| lymph node metastases | 30 | 0.27 ± 0.09 |
| Visceral metastases | 46 | 0.28 ± 0.06 |

*Data are shown as mean ± SEM.

TABLE 4
Clinical data of the Australia melanoma study, Related to FIG. 2

| | n | % |
|---|---|---|
| Total | 70 | |
| Sex | | |
| Male | 46 | 65.7 |
| Female | 24 | 34.3 |
| Mean Age (range) | 60.0 (27-89) | |
| Survival at follow-up | | |
| Alive | 32 | 45.7 |
| Deceased | 38 | 54.3 |

TABLE 5
Clinically annotated cohort study: Percent of cells staining
positively for 5-hmC stratified by Breslow depth, metastases,
overall survival, and stage, Related to FIG. 2

| | Positivity of 5-hmC stain (cell count %) | | | | |
|---|---|---|---|---|---|
| Group | <1% | 1-9% | 10-24% | 25-74% | >75% |
| Breslow >1 | 7.07% | 39.90% | 13.64% | 26.26% | 13.13% |
| Breslow ≦1 | 8.33% | 0.00% | 0.00% | 41.67% | 50.00% |
| Developed metastases | 10.61% | 35.61% | 11.36% | 30.30% | 12.12% |
| No metastases | 1.28% | 41.03% | 15.38% | 21.80% | 20.51% |
| Alive at follow-up | 4.17% | 35.42% | 15.62% | 21.87% | 22.92% |
| Deceased | 9.65% | 39.47% | 10.53% | 31.58% | 8.77% |
| Stage 1 | 4.76% | 14.29% | 14.29% | 33.33% | 33.33% |
| Stages 2-3 | 7.40% | 40.21% | 12.70% | 25.46% | 13.23% |

TABLE 6
5-hmC staining analyses of the Australian
melanoma cohort study, Related to FIG. 2

| | n | Staining Score# | P-value (t-test) |
|---|---|---|---|
| Breslow Depth | | | 0.008 ^{A} |
| ≦1 mm | 4 | 4.97 ± 1.62 | |
| >1 mm | 66 | 1.71 ± 0.28 | |
| Stage | | | 0.0110 ^{B} |
| 1 | 7 | 4.08 ± 1.22 | |
| 2-3 | 63 | 1.65 ± 0.28 | |
| Mitoses | | | 0.0134 ^{C} |
| ≦1 mitosis | 14 | 3.97 ± 0.890 | |
| >1 mitosis | 56 | 1.37 ± 0.25 | |
| Ulceration | | | 0.0327^{D} |
| Without ulceration | 38 | 2.34 ± 0.47 | |
| With ulceration | 26 | 1.0 ± 0.28 | |

^{A} P < 0.05 (Breslow ≦1 mm vs Breslow >1 mm);
^{B} P < 0.05 (Stage 1 vs Stage 2-3);
^{C} P < 0.01 (≦1 mitosis vs >1 mitosis);
^{D}P < 0.05 (without vs with ulceration).
#5-hmC staining score = score of cell count × score of intensity.
Data given as mean ± SEM.

TABLE 7
hMeDIP-qPCR primers, Related to FIGS. 3 and 5

| Primer | Sequence | |
|---|---|---|
| MC1R F | ACCAGGGCTTTGGCCTTAAA | (SEQ ID NO: 1) |
| MC1R R | ATTGCAGATGATGAGGGCGA | (SEQ ID NO: 2) |
| CCND1 F | ATTTCCAATCCGCCCTCCAT | (SEQ ID NO: 3) |
| CCND1 R | TCACTTACCGGGTCACACTTGA | (SEQ ID NO: 4) |
| IGF1R F | AGTGGGAAGCATGGAAGCAT | (SEQ ID NO: 5) |
| IGF1R R | ACAGCTCAGCAGCCAAGTAT | (SEQ ID NO: 6) |
| RAC3 F | TTCTGTGCAGACTTGTGAACCC | (SEQ ID NO: 7) |
| RAC3 R | ACCATCACGTTGGCAGAGTA | (SEQ ID NO: 8) |
| TIMP2 F | TCGATGTCGAGAAACTCCTGCT | (SEQ ID NO: 9) |
| TIMP2 R | AAGAACATCAACGGGCACCA | (SEQ ID NO: 10) |

TABLE 8
RT-qPCR primers, Related to FIG. 4

| Primer | Sequence | |
|---|---|---|
| IDH1 F | TCCGTCACTTGGTGTGTAGG | (SEQ ID NO: 11) |
| IDH1 R | GGCTTGTGAGTGGATGGGTA | (SEQ ID NO: 12) |
| IDH2 F | TGAACTGCCAGATAATACGGG | (SEQ ID NO: 13) |
| IDH2 R | CTGACAGCCCCCACCTC | (SEQ ID NO: 14) |
| CTSB F | GACAGGGGATGGAAAGAGG | (SEQ ID NO: 15) |
| CTSB R | TGGTTTGCATAGATGATTGGC | (SEQ ID NO: 16) |
| TET1 F | GCTATACACAGAGCTCACAG | (SEQ ID NO: 17) |
| TET1 R | GCCAAAAGAGAATGAAGCTCC | (SEQ ID NO: 18) |
| TET2 F | CTTTCCTCCCTGGAGAACAGCTC | (SEQ ID NO: 19) |
| TET2 R | TGCTGGGACTGCTGCATGACT | (SEQ ID NO: 20) |
| TET3 F | GTTCCTGGAGCATGTACTTC | (SEQ ID NO: 21) |
| TET3 R | CTTCCTCTTTGGGATTGTCC | (SEQ ID NO: 22) |
| GAPDH F | GAAGGTGAAGGTCGGAGTC | (SEQ ID NO: 23) |
| GAPDH R | GAAGATGGTGATGGGATTTC | (SEQ ID NO: 24) |
| HPRT F | GACTTTGCTTTCCTTGGTC | (SEQ ID NO: 25) |
| HPRT R | AGTCAAGGGCATATCCTAC | (SEQ ID NO: 26) |
| PTEN F | GGTTGCCACAAAGTGCCTCGTTTA | (SEQ ID NO: 27) |
| PTEN R | AACTGGCAGGTAGAAGGCAACTCT | (SEQ ID NO: 28) |

### REFERENCES

Ceol, C. J., Houvras, Y., Jane-Valbuena, J., Bilodeau, S., Orlando, D. A., Battisti, V., Fritsch, L., Lin, W. M., Hollmann, T. J., Ferre, F., et al. (2011). The histone methyltransferase SETDB1 is recurrently amplified in melanoma and accelerates its onset. Nature 471, 513-517.
Chowdhury, R., Yeoh, K. K., Tian, Y. M., Hillringhaus, L., Bagg, E. A., Rose, N. R., Leung, I. K., Li, X. S., Woon, E. C., Yang, M., et al. (2011). The oncometabolite 2-hydroxyglutarate inhibits histone lysine demethylases. EMBO Rep 12, 463-469.
Cimmino, L., Abdel-Wahab, O., Levine, Ross L., and Aifantis, I. (2011). TET Family Proteins and Their Role in Stem Cell Differentiation and Transformation. Cell stem cell 9, 193-204.
Conway, K., Edmiston, S. N., Khondker, Z. S., Groben, P. A., Zhou, X., Chu, H., Kuan, P. F., Hao, H., Carson, C., Berwick, M., et al. (2011). DNA-methylation profiling distinguishes malignant melanomas from benign nevi. Pigment cell & melanoma research 24, 352-360.
Dang, L., Jin, S., and Su, S. M. (2010). IDH mutations in glioma and acute myeloid leukemia. Trends Mol Med 16, 387-397.
Delhommeau, F., Dupont, S., Della Valle, V., James, C., Trannoy, S., Masse, A., Kosmider, O., Le Couedic, J. P., Robert, F., Alberdi, A., et al. (2009). Mutation in TET2 in myeloid cancers. The New England journal of medicine 360, 2289-2301.
Ficz, G., Branco, M. R., Seisenberger, S., Santos, F., Krueger, F., Hore, T. A., Marques, C. J., Andrews, S., and Reik, W. (2011). Dynamic regulation of 5-hydroxymethylcytosine in mouse ES cells and during differentiation. Nature 473, 398-402.
Haffner, M. C., Chaux, A., Meeker, A. K., Esopi, D. M., Gerber, J., Pellakuru, L. G., Toubaji, A., Argani, P., Iacobuzio-Donahue, C., Nelson, W. G., et al. (2011). Global 5-hydroxymethylcytosine content is significantly reduced in tissue stem/progenitor cell compartments and in human cancers. Oncotarget.
Haqq, C., Nosrati, M., Sudilovsky, D., Crothers, J., Khodabakhsh, D., Pulliam, B. L., Federman, S., Miller, J. R., Allen, R. E., Singer, M. I., et al. (2005). The gene expression signatures of melanoma progression. Proc Natl Acad Sci USA 102, 6092-6097.
Hoon, D. S., Spugnardi, M., Kuo, C., Huang, S. K., Morton, D. L., and Taback, B. (2004). Profiling epigenetic inactivation of tumor suppressor genes in tumors and plasma from cutaneous melanoma patients. Oncogene 23, 4014-4022.
Ito, S., D'Alessio, A. C., Taranova, O. V., Hong, K., Sowers, L. C., and Zhang, Y. (2010). Role of Tet proteins in 5mC to 5hmC conversion, ES-cell self-renewal and inner cell mass specification. Nature 466, 1129-1133.
Jemal, A., Devesa, S. S., Hartge, P., and Tucker, M. A. (2001). Recent trends in cutaneous melanoma incidence among whites in the United States. J Natl Cancer Inst 93, 678-683.
Jemal, A., Siegel, R., Ward, E., Murray, T., Xu, J., Smigal, C., and Thun, M. J. (2006). Cancer statistics, 2006. CA: a cancer journal for clinicians 56, 106-130.
Kabbarah, O., Nogueira, C., Feng, B., Nazarian, R. M., Bosenberg, M., Wu, M., Scott, K. L., Kwong, L. N., Xiao, Y., Cordon-Cardo, C., et al. (2010). Integrative genome comparison of primary and metastatic melanomas. PloS one 5, e10770.
Krell, D., Assoku, M., Galloway, M., Mulholland, P., Tomlinson, I., and Bardella, C. (2011). Screen for IDH1, IDH2, IDH3, D2HGDH and L2HGDH mutations in glioblastoma. PloS one 6, e19868.
Langemeijer, S. M., Kuiper, R. P., Berends, M., Knops, R., Aslanyan, M. G., Massop, M., Stevens-Linders, E., van Hoogen, P., van Kessel, A. G., Raymakers, R. A., et al. (2009). Acquired mutations in TET2 are common in myelodysplastic syndromes. Nature genetics 41, 838-842.
Liu, S., Ren, S., Howell, P., Fodstad, O., and Riker, A. I. (2008). Identification of novel epigenetically modified genes in human melanoma via promoter methylation gene profiling. Pigment cell & melanoma research 21, 545-558.
Marks, R. (2000). Epidemiology of melanoma. Clin Exp Dermatol 25, 459-463.
Paz, M. F., Fraga, M. F., Avila, S., Guo, M., Pollan, M., Herman, J. G., and Esteller, M. (2003). A systematic profile of DNA methylation in human cancer cell lines. Cancer research 63, 1114-1121.
Reitman, Z. J., Jin, G., Karoly, E. D., Spasojevic, I., Yang, J., Kinzler, K. W., He, Y., Bigner, D. D., Vogelstein, B., and Yan, H. (2011). Profiling the effects of isocitrate dehydrogenase 1 and 2 mutations on the cellular metabolome. Proceedings of the National Academy of Sciences of the United States of America 108, 3270-3275.
Riker, A., Enkemann, S., Fodstad, 0., Liu, S., Ren, S., Morris, C., Xi, Y., Howell, P., Metge, B., Samant, R., et al. (2008). The gene expression profiles of primary and metastatic melanoma yields a transition point of tumor progression and metastasis. BMC Medical Genomics 1, 13.
Schatton, T., Murphy, G. F., Frank, N. Y., Yamaura, K., Waaga-Gasser, A. M., Gasser, M., Zhan, Q., Jordan, S., Duncan, L. M., Weishaupt, C., et al. (2008). Identification of cells initiating human melanomas. Nature 451, 345-349.
Shen, L., Kondo, Y., Guo, Y., Zhang, J., Zhang, L., Ahmed, S., Shu, J., Chen, X., Waterland, R. A., and Issa, J. P. (2007). Genome-wide profiling of DNA methylation reveals a class of normally methylated CpG island promoters. PLoS Genet 3, 2023-2036.
Song, C. X., Szulwach, K. E., Fu, Y., Dai, Q., Yi, C., Li, X., Li, Y., Chen, C. H., Zhang, W., Jian, X., et al. (2011). Selective chemical labeling reveals the genome-wide distribution of 5-hydroxymethylcytosine. Nat Biotechnol 29, 68-72.
Szwagierczak, A., Bultmann, S., Schmidt, C. S., Spada, F., and Leonhardt, H. (2010). Sensitive enzymatic quantification of 5-hydroxymethylcytosine in genomic DNA. Nucleic Acids Res 38, e181.
Tahiliani, M., Koh, K. P., Shen, Y., Pastor, W. A., Bandukwala, H., Brudno, Y., Agarwal, S., Iyer, L. M., Liu, D. R., Aravind, L., et al. (2009). Conversion of 5-methylcytosine to 5-hydroxymethylcytosine in mammalian DNA by MLL partner TET1. Science 324, 930-935.
Talantov, D., Mazumder, A., Yu, J. X., Briggs, T., Jiang, Y., Backus, J., Atkins, D., and Wang, Y. (2005). Novel Genes Associated with Malignant Melanoma but not Benign Melanocytic Lesions. Clinical Cancer Research 11, 7234-7242.
Tellez, C. S., Shen, L., Estecio, M. R., Jelinek, J., Gershenwald, J. E., and Issa, J.-P. J. (2009). CpG island methylation profiling in human melanoma cell lines. Melanoma Research 19, 146-155.
Ward, P. S., Patel, J., Wise, D. R., Abdel-Wahab, O., Bennett, B. D., Coller, H. A., Cross, J. R., Fantin, V. R., Hedvat, C. V., Perl, A. E., et al. (2010). The Common Feature of Leukemia-Associated IDH1 and IDH2 Mutations Is a Neomorphic Enzyme Activity Converting Î±-Ketoglutarate to 2-Hydroxyglutarate. Cancer Cell 17, 225-234.
Wrzeszczynski, K. O., Varadan, V., Byrnes, J., Lum, E., Kamalakaran, S., Levine, D. A., Dimitrova, N., Zhang, M. Q., and Lucito, R. (2011). Identification of Tumor Suppressors and Oncogenes from Genomic and Epigenetic Features in Ovarian Cancer. PLoS ONE 6, e28503.
Wu, H., and Zhang, Y. (2011). Mechanisms and functions of Tet protein-mediated 5-methylcytosine oxidation. Genes & Development 25, 2436-2452.
Xu, W., Yang, H., Liu, Y., Yang, Y., Wang, P., Kim, S.-H., Ito, S., Yang, C., Wang, P., Xiao, M.-T., et al. (2011a). Oncometabolite 2-Hydroxyglutarate Is a Competitive Inhibitor of alpha-Ketoglutarate-Dependent Dioxygenases. Cancer cell 19, 17-30.
Xu, Y., Wu, F., Tan, L., Kong, L., Xiong, L., Deng, J., Barbera, A. J., Zheng, L., Zhang, H., Huang, S., et al. (2011b). Genome-wide Regulation of 5hmC, 5mC, and Gene Expression by Tet1 Hydroxylase in Mouse Embryonic Stem Cells. Mol Cell 42, 451-464.
Yang, H., Liu, Y., Bai, F., Zhang, J. Y., Ma, S. H., Liu, J., Xu, Z. D., Zhu, H. G., Ling, Z. Q., Ye, D., et al. (2012). Tumor development is associated with decrease of TET gene expression and 5-methylcytosine hydroxylation. Oncogene.
Zhang, H., Zhang, X., Clark, E., Mulcahey, M., Huang, S., and Shi, Y. G. (2010). TET1 is a DNA-binding protein that modulates DNA methylation and gene transcription via hydroxylation of 5-methylcytosine. Cell Res 20, 1390-1393.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.

1.-30. (canceled)

31. A method of diagnosing a subject with a melanocytic lesion comprising,
a) processing a tissue sample of the melanocytic lesion of the subject to thereby label 5-hmC present in the tissue sample;
b) measuring the 5-hmC in the sample by detection of the labeled 5-hmC in the tissue sample;
c) quantitating the 5-hmC in the tissue sample as compared to a healthy control to thereby detect a threshold level of reduction of 5-hmC in the sample; and
d) characterizing the melanocytic lesion by the detected threshold level of reduction of 5-hmC to thereby diagnose the subject.

32. The method of claim 31, wherein the processing step a) is by immunohistochemical staining or by immunofluorescence and/or wherein quantitating step c) is by assignment of a 5-hmC staining score or by assignment of a 5-hmC cell count score.

33. The method of claim 31, wherein the threshold level of reduction is equivalent to:
a) a 5-hmC staining score of ≦2 which indicates the melanocytic lesion is a stage 2-3 melanoma, with a Breslow of >1 mm, and >1 mitosis;
b) a 5-hmC staining score of ≧3 which indicates the melanocytic lesion is a stage 1 melanoma, with a Breslow of ≦1 mm, and ≦1 mitosis;
c) a 5-hmC staining score of ≧1.5 which indicates the melanocytic lesion is a melanoma without ulceration; or
d) a 5-hmC staining score of <1.5 which indicates the melanocytic lesion is a melanoma with ulceration.

34. The method of claim 31 wherein the threshold level of reduction is equivalent to:
a) a 5-hmC cell count score of ≧2 which indicates the melanocytic lesion is a benign melanocytic nevus;
b) a 5-hmC cell count score of ≧3 which indicates the melanocytic lesion is a benign melanocytic nevus;
c) a 5-hmC cell count score of <1 which indicates the melanocytic lesion is a primary cutaneous melanoma or a visceral metastatsic melanoma;
d) a 5-hmC cell count score of <0.5 which indicates the melanocytic lesion is a primary cutaneous melanoma or a visceral metastatsic melanoma; or
e) a 5-hmC cell count score of <0.25 which indicates the melanocytic lesion is a lymphnode metastatic melanoma.

35. The method of claim 31, wherein processing step a) is by purifying genomic DNA from a tissue sample of the melanocytic lesion and measuring step b) is by sequencing genomic DNA identified as containing 5-hmC in the purified genomic DNA or by detection of the 5-hmC in specific genes of the genomic DNA, or by an anti-5-hmC antibody-based detection system.

36. The method of claim 35, wherein the threshold level of reduction is a ≧5 fold reduction in the 5-hmC of the specific genes of the genomic DNA which indicates the melanocytic lesion is a melanoma.

37. The method of claim 35, wherein the anti-5-hmC antibody-based detection system is a dot blot assay and/or the 5-hmC levels are detected by a 5-hmC glucosylation assay.

38. The method of claim 37 wherein the 5-hmC glucosylation assay is a T4 phage β-glucosyltransferase-mediated 5-hmC glucosylation assay.

39. A method of diagnosing a subject having a melanocytic lesion comprising,
a) processing a tissue sample of the melanocytic lesion of the subject to thereby label expression product of one or more of the genes IDH2, TET1, TET2, TET3;
b) measuring the expression product of the one or more genes in the sample by detection of the labeled expression product in the tissue sample;
c) quantitating the labeled expression product(s) in the sample as compared to a healthy control to thereby detect a threshold level of reduction of the expression product(s) in the sample; and
d) diagnosing the subject as having a malignancy if a level of reduction of TET3 and/or IDH2 of >50% is detected, and/or if a level of reduction of TET1 and/or TET2 of >75% is detected.

40. The method of claim 39 further comprising treating a subject diagnosed with a melanoma comprising contacting melanoma cells of the subject with an effective amount of an agent that increases expression of IDH2 and/or TET2 sufficient to increase 5-hmC in the genome of the cell.

41. The method of claim 40, wherein the agent is selected from an expression vector encoding IDH2 and/or TET2, a regulatory molecule which increases transcription or translation of the IDH2 and/or TET2 gene, and combinations thereof.

42. The method of claim 40, wherein contacting is by administering to the subject a therapeutic amount of a pharmaceutical composition comprising the agent.

43. The method of claim 42, wherein administering is intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, or intratumor.

44. A method of determining prognosis of a subject with a melanocytic lesion comprising,
a) processing a tissue sample of the melanocytic lesion of the subject to thereby label the 5-hmC present in the tissue sample;
b) measuring the 5-hmC in the sample by detection of the labeled 5-hmC in the tissue sample;
c) quantitating the 5-hmC in the tissue sample as compared to a healthy control to thereby detect a threshold level of reduction of 5-hmC in the sample;
d) correlating the threshold level of reduction detected in step c) with one or more melanoma staging parameters; and
e) determining the prognosis of the subject based on that of the staging parameter to which the amount of 5-hmC is correlated.

45. The method of claim 44, wherein the staging parameter is selected from the group consisting of Breslow depth, mitosis rate, presence or absence of ulceration, overall stage of melanoma, melanocytic lesion type, and combinations thereof.

46. The method of claim 44, wherein the processing step a) is by immunohistochemical staining or by immunofluorescence.

47. The method of claim 46, wherein quantitating step c) is by assignment of a 5-hmC staining score.

48. The method of claim 44, wherein the threshold level of reduction is equivalent to:
a) a 5-hmC staining score of ≦2 which indicates the melanocytic lesion is a stage 2-3 melanoma, with a Breslow of >1 mm, and >1 mitosis;
b) a 5-hmC staining score of ≧3 which indicates the melanocytic lesion is a stage 1 melanoma, with a Breslow of ≦1 mm, and ≦1 mitosis;
c) a 5-hmC staining score of ≧1.5 which indicates the melanocytic lesion is a melanoma without ulceration; or
d) a 5-hmC staining score of <1.5 which indicates the melanocytic lesion is a melanoma with ulceration.

49. The method of claim 46, wherein quantitating step c) is by assignment of a 5-hmC cell count score.

50. The method of claim 44, wherein the threshold level of reduction is equivalent to:
a) a 5-hmC cell count score of ≧2 which indicates the melanocytic lesion is a benign melanocytic nevus;
b) a 5-hmC cell count score of ≧3 which indicates the melanocytic lesion is a benign melanocytic nevus;
c) a 5-hmC cell count score of <1 which indicates the melanocytic lesion is a primary cutaneous melanoma or a visceral metastatsic melanoma;
d) a 5-hmC cell count score of <0.5 which indicates the melanocytic lesion is a primary cutaneous melanoma or a visceral metastatsic melanoma; or
e) a 5-hmC cell count score of <0.25 which indicates the melanocytic lesion is a lymphnode metastatic melanoma.


SEQUENCE LISTING (28 sequences)

SEQ ID NO: 1 - DNA, 20 bp, Artificial Sequence
  Description of Artificial Sequence Synthetic primer
accagggctttggccttaaa

SEQ ID NO: 2 - DNA, 20 bp, Artificial Sequence
  Description of Artificial Sequence Synthetic primer
attgcagatgatgagggcga

SEQ ID NO: 3 - DNA, 20 bp, Artificial Sequence
  Description of Artificial Sequence Synthetic primer
atttccaatccgccctccat

SEQ ID NO: 4 - DNA, 22 bp, Artificial Sequence
  Description of Artificial Sequence Synthetic primer
tcacttaccgggtcacacttga

SEQ ID NO: 5 - DNA, 20 bp, Artificial Sequence
  Description of Artificial Sequence Synthetic primer
agtgggaagcatggaagcat

SEQ ID NO: 6 - DNA, 20 bp, Artificial Sequence
  Description of Artificial Sequence Synthetic primer
acagctcagcagccaagtat

SEQ ID NO: 7 - DNA, 22 bp, Artificial Sequence
  Description of Artificial Sequence Synthetic primer
ttctgtgcagacttgtgaaccc

SEQ ID NO: 8 - DNA, 20 bp, Artificial Sequence
  Description of Artificial Sequence Synthetic primer
accatcacgttggcagagta

SEQ ID NO: 9 - DNA, 22 bp, Artificial Sequence
  Description of Artificial Sequence Synthetic primer
tcgatgtcgagaaactcctgct

SEQ ID NO: 10 - DNA, 20 bp, Artificial Sequence
  Description of Artificial Sequence Synthetic primer
aagaacatcaacgggcacca

SEQ ID NO: 11 - DNA, 20 bp, Artificial Sequence
  Description of Artificial Sequence Synthetic primer
tccgtcacttggtgtgtagg

SEQ ID NO: 12 - DNA, 20 bp, Artificial Sequence
  Description of Artificial Sequence Synthetic primer
ggcttgtgagtggatgggta

SEQ ID NO: 13 - DNA, 21 bp, Artificial Sequence
  Description of Artificial Sequence Synthetic primer
tgaactgccagataatacggg

SEQ ID NO: 14 - DNA, 17 bp, Artificial Sequence
  Description of Artificial Sequence Synthetic primer
ctgacagcccccacctc

SEQ ID NO: 15 - DNA, 19 bp, Artificial Sequence
  Description of Artificial Sequence Synthetic primer
gacaggggatggaaagagg

SEQ ID NO: 16 - DNA, 21 bp, Artificial Sequence
  Description of Artificial Sequence Synthetic primer
tggtttgcatagatgattggc

SEQ ID NO: 17 - DNA, 20 bp, Artificial Sequence
  Description of Artificial Sequence Synthetic primer
gctatacacagagctcacag

SEQ ID NO: 18 - DNA, 21 bp, Artificial Sequence
  Description of Artificial Sequence Synthetic primer
gccaaaagagaatgaagctcc

SEQ ID NO: 19 - DNA, 23 bp, Artificial Sequence
  Description of Artificial Sequence Synthetic primer
ctttcctccctggagaacagctc

SEQ ID NO: 20 - DNA, 21 bp, Artificial Sequence
  Description of Artificial Sequence Synthetic primer
tgctgggactgctgcatgact

SEQ ID NO: 21 - DNA, 20 bp, Artificial Sequence
  Description of Artificial Sequence Synthetic primer
gttcctggagcatgtacttc

SEQ ID NO: 22 - DNA, 20 bp, Artificial Sequence
  Description of Artificial Sequence Synthetic primer
cttcctctttgggattgtcc

SEQ ID NO: 23 - DNA, 19 bp, Artificial Sequence
  Description of Artificial Sequence Synthetic primer
gaaggtgaaggtcggagtc

SEQ ID NO: 24 - DNA, 20 bp, Artificial Sequence
  Description of Artificial Sequence Synthetic primer
gaagatggtgatgggatttc

SEQ ID NO: 25 - DNA, 19 bp, Artificial Sequence
  Description of Artificial Sequence Synthetic primer
gactttgctttccttggtc

SEQ ID NO: 26 - DNA, 19 bp, Artificial Sequence
  Description of Artificial Sequence Synthetic primer
agtcaagggcatatcctac

SEQ ID NO: 27 - DNA, 24 bp, Artificial Sequence
  Description of Artificial Sequence Synthetic primer
ggttgccacaaagtgcctcgttta

SEQ ID NO: 28 - DNA, 24 bp, Artificial Sequence
  Description of Artificial Sequence Synthetic primer
aactggcaggtagaaggcaactct